(19) United States
(12) Patent Application Publication
(10) Pub. No.: US 2018/0066081 A1
(43) Pub. Date: Mar. 8, 2018

(54) SUBSTITUTED CYCLIC ETHER POLYMERS AND CONJUGATES AND USES THEREOF

(71) Applicant: UNIVERSITY OF IOWA RESEARCH FOUNDATION, Iowa City, IA (US)

(72) Inventor: Russell P. PESAVENTO, Chicago, IL (US)

(21) Appl. No.: 15/548,693

(22) PCT Filed: Feb. 9, 2016
(86) PCT No.: PCT/US2016/017124

§ 371 Date: Aug. 3, 2017

Related U.S. Application Data
(60) Provisional application No. 62/113,983, filed on Feb. 9, 2015.

(51) Int. Cl. C08F 34/02; A61K 31/765; A61K 47/58; C07D 307/28; C07D 405/12; A61K 31/80; A61K 31/4164

(52) U.S. Cl. CPC C08F 34/02; A61K 31/765; A61K 47/58; A61K 31/4164; C07D 405/12; A61K 31/80; C07D 307/28

(57) ABSTRACT

Disclosed herein are substituted cyclic ether monomers, polymers, and drug conjugates thereof, which are useful for the treatment of diseases and conditions of the oral cavity. In particular, disclosed herein are monomers of Formula (I), polymers of Formula (II), drug conjugates of the monomers and polymers, and pharmaceutically acceptable salts and solvates of each:

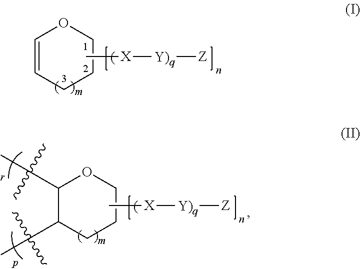

wherein the substituents are as described.

### CROSS-REFERENCE TO RELATED APPLICATION

This application is the National Stage of International Application No. PCT/US2016/017124, filed Feb. 9, 2016, which claims priority to U.S. Provisional Patent Application Ser. No. 62/113,983, filed Feb. 9, 2015, the entire disclosures of which are incorporated herein by reference.

### BACKGROUND

### Technical Field

The present disclosure relates to substituted cyclic ether monomers, polymers, and drug conjugates thereof, and methods of using the polymers and drug conjugates in, e.g., the treatment of oral diseases and conditions, such as oral mucositis, microbial infections, and cancer.

### Description of Related Technology

Oral diseases and conditions, such as microbial based infections (e.g., gingivitis, periodontitis, necrotizing ulcerative gingivitis, necrotizing ulcerative periodontitis, periocoronitis, dental caries, abscessed teeth, and cellulitis), mucositis (i.e., stomatitis), and various head and neck cancers affect every population and age group. See, e.g., Neville, D.; Damm, D. D.; Allen, C. M; Bouqout, J. E. “Oral and Maxillofacial Pathology, Third Edition, 2009. To combat these diseases and conditions, medical clinicians often prescribe either systemic antibiotics (e.g., penicillin VK, amoxicillin, or clindamycin) and/or systemic analgesics/anti-inflammatories (e.g., NSAIDs like ibuprofen) to comfort the patient and allow sufficient time for the patient to seek further dental care, such as oral surgery, endodontic therapy, or restorative dental work. However, repeated systemic dosing of well-known antibiotics typically requires higher and higher doses for continual effectiveness in and around the oral cavity. This continued high dosing of systemic antibiotics elevates the risk of future microbial resistance and diminishes the already short list of effective antibiotics. Additionally, the continued high doses of systemic anti-inflammatory medications (e.g., NSAIDs) required to combat the pain associated with oral diseases and conditions often results in gastrointestinal discomfort for patients. This discomfort is exasperated by the fact that commonly prescribed doses of NSAIDs are associated with adverse reactions in patients with non-oral medical conditions, such as kidney failure, stomach ulcers, and pregnancies. See Mosby's Dental Drug Reference, 2010, Ninth Edition.

Unlike medical clinicians, who often only prescribe antibiotics and analgesics/anti-inflammatories to treat oral diseases and conditions, dentists also often prescribe topical therapeutics or rinses. However, these remedies have a limited therapeutic window of activity in the oral cavity. For example, chlorhexidine rinses (e.g., PERIDEX), which are prescribed for various pathogenic bacterial infections of the oral cavity, only result in about 30% of the active compound (chlorhexidine) being retained in the oral cavity following application. Further, the low amount of chlorhexidine that is retained often results in undesirable tooth staining. See PERIDEX (chlorhexidine gluconoate 0.12%) package insert. St. Paul, Minn.: 3M ESPE Dental Products; 2013. Retaining topical therapeutics and rinses in the oral cavity for a therapeutically effective time period also is significantly challenged by the harsh environment of the oral cavity due to saliva, food consumption, and mechanical forces (e.g., chewing, teeth brushing).

In recent years, much effort has focused on topical applications for controlled release of medicaments in the oral cavity. A common approach for local oral delivery of antibacterial, antifungal, or anesthetic agents is through the non-covalent trapping of the therapeutic agents in crosslinked polymeric matrices known as hydrogels or microspheres (e.g., PERICHIP and ARRESTIN). Over time, the therapeutic agent can diffuse out of the matrix and into oral tissue, causing a therapeutic effect. Controlling the rate of diffusion of the therapeutic agent out of the crosslinked matrices, however, can be difficult. Furthermore, many matrices must be extensively crosslinked to result in slow enough release of the therapeutic agent into the oral cavity, which compromises the ability of the matrix to adhere to oral tissues. See Salamat-Miller, N.; et al. “Adv. Drug. Deliv. Rev., 2005, 1661-1691. Therefore, diffusion-controlled delivery of therapeutic agents into the oral cavity can have the disadvantages of a limited lifetime and poor adhesion to the oral cavity.

Mucoadhesive films, which are composed of polymers that interact with the carbohydrates and glycoproteins that line non-keratinized epithelial surfaces in the oral cavity, also have been investigated for the treatment of oral conditions, such as oral lesions, by offering palliative relief. These mucoadhesive films are generally composed of a polymer system, which includes various adjuvants that can adhere to the oral mucosa for an extended period of time. Two examples of these mucoadhesive films include MUGARD and GELClAIR. Although these mucoadhesive films can be useful in offering palliative relief, they do not offer any therapeutic relief.

Thus, a need exists for new compounds that are capable of both adhering to the oral mucosa for significant periods of time and delivering therapeutic agents to the oral cavity, such as through controlled release.

### SUMMARY

One aspect of the disclosure provides a polymer having a structure of Formula (II), or a pharmaceutically acceptable salt or solvate thereof:

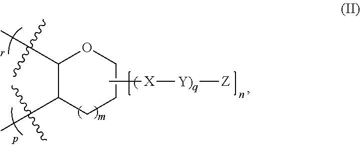

wherein:
p+r is 2 to 500;
m is 0 or 1;
when m is 0, then n is 1 or 2, and when m is 1 then n is 1, 2, or 3;
each q is 0 or 1;
each X independently is C₁₋₁₀alkylene, arylene, C₂₋₁₀polyalkyleneoxide, or absent;
each Y independently is O, S, or NR¹;
when q is 0, then each Z independently is C₁₋₁₀alkyl, C₀₋₁₀alkylene-C₃₋₈cycloalkyl, C₀₋₁₀alkylene-C₃₋₈heterocycloalkyl, C₀₋₁₀alkylene-aryl, C₀₋₁₀alkylene-heteroaryl, C₀₋₁₀alkylene-COOR¹, C₀₋₁₀alkylene-CN, C₀₋₁₀alkylene-NR¹₃⁺, C₀₋₁₀alkylene-ONO₂, C₀₋₁₀alkylene-OSO₂R², C₀₋₁₀alkylene-OPO₃H₂, C₂₋₁₀polyalkyleneoxide, C₂₋₁₀polyalkyleneoxide-C₃₋₈cycloalkyl, C₂₋₁₀polyalkyleneoxide-C₃₋₈heterocycloalkyl, C₂₋₁₀polyalkyleneoxide-aryl, C₂₋₁₀polyalkyleneoxide-heteroaryl, C₂₋₁₀polyalkyleneoxide-COOR¹, C₂₋₁₀polyalkyleneoxide-CN, C₂₋₁₀polyalkyleneoxide-NR¹₂, C₂₋₁₀polyalkyleneoxide-NR¹₃⁺, C₂₋₁₀polyalkyleneoxide-ONO₂, C₂₋₁₀polyalkyleneoxide-OSO₂R², C₂₋₁₀polyalkyleneoxide-OPO₃H₂, or

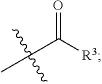

when q is 1, then each Z independently is H, C₀₋₁₀alkylene-C₃₋₈cycloalkyl, C₀₋₁₀alkylene-C₃₋₈heterocycloalkyl, C₀₋₁₀alkylene-aryl, C₀₋₁₀alkylene-heteroaryl, C₀₋₁₀alkylene-COOR¹, C₁₋₁₀alkylene-CN, C₁₋₁₀alkylene-OR¹, C₁₋₁₀alkylene-SR¹, C₁₋₁₀alkylene-NR¹₂, C₁₋₁₀alkylene-NR¹₃⁺, C₁₋₁₀alkylene-ONO₂, C₁₋₁₀alkylene-OSO₂R², C₁₋₁₀alkylene-OPO₃H₂, C₂₋₁₀polyalkyleneoxide, C₂₋₁₀polyalkyleneoxide-C₃₋₈cycloalkyl, C₂₋₁₀polyalkyleneoxide-C₃₋₈heterocycloalkyl, C₂₋₁₀polyalkyleneoxide-aryl, C₂₋₁₀polyalkyleneoxide-heteroaryl, C₂₋₁₀polyalkyleneoxide-COOR¹, C₂₋₁₀polyalkyleneoxide-CN, C₂₋₁₀polyalkyleneoxide-OR¹, C₂₋₁₀polyalkyleneoxide-SR¹, C₂₋₁₀polyalkyleneoxide-NR¹₂, C₂₋₁₀polyalkyleneoxide-NR¹₃⁺, C₂₋₁₀polyalkyleneoxide-ONO₂, C₂₋₁₀polyalkyleneoxide-OSO₂R², C₂₋₁₀polyalkyleneoxide-OPO₃H₂, or

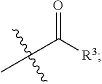

each R¹ independently is H, C₁₋₆alkyl, or aryl;
each R² independently is OR¹ or NR¹₂;
each R³ independently is C₁₋₁₀alkyl, C₀₋₁₀alkylene-C₃₋₈cycloalkyl, C₀₋₁₀alkylene-C₅₋₈heterocycloalkyl, C₀₋₁₀alkylene-aryl, C₀₋₁₀alkylene-heteroaryl, C₁₋₁₀alkylene-COOR⁴, C₀₋₁₀alkylene-OR⁴, C₀₋₁₀alkylene-SR⁴, C₀₋₁₀alkylene-NR¹₂, C₀₋₁₀alkylene-NR¹₃⁺, C₁₋₁₀alkylene-CN, C₁₋₁₀alkylene-NO₂, C₁₋₁₀alkylene-ONO₂, C₁₋₁₀alkylene-SO₂R², C₁₋₁₀alkylene-OSO₂R², C₀₋₁₀alkylene-PO₃H₂, C₀₋₁₀alkylene-OPO₃H₂, C₂₋₁₀polyalkyleneoxide, C₂₋₁₀polyalkyleneoxide-C₃₋₈cycloalkyl, C₂₋₁₀polyalkyleneoxide-C₅₋₈heterocycloalkyl, C₂₋₁₀polyalkyleneoxide-aryl, C₂₋₁₀polyalkyleneoxide-heteroaryl, C₂₋₁₀polyalkyleneoxide-COOR⁴, C₂₋₁₀polyalkyleneoxide-OR⁴, C₂₋₁₀polyalkyleneoxide-SR⁴, C₂₋₁₀polyalkyleneoxide-NR¹₂, C₂₋₁₀polyalkyleneoxide-NR¹₃⁺, C₂₋₁₀polyalkyleneoxide-CN, C₂₋₁₀polyalkyleneoxide—NO₂, C₂₋₁₀polyalkyleneoxide-SO₃R⁴, C₂₋₁₀polyalkyleneoxide-PO₃H₂, or CH(NR¹₂)R⁵;
each R⁴ independently is H, C₁₋₁₀alkyl, or C₂₋₁₀polyalkyleneoxide; and
each R⁵ independently is H, C₁₋₆alkyl, C₁₋₆alkylene-OR¹, C₁₋₆alkylene-SR¹, C₁₋₆alkylene-COOH, C₁₋₆alkylene-CONR¹₂, C₁₋₆alkylene-NR¹₂, C₁₋₆alkylene-NHC(NH₂)₂, C₁₋₆alkylene-aryl, or C₁₋₆alkylene-heteroaryl.

Another aspect of the disclosure provides a monomer of Formula (I), or a pharmaceutically acceptable salt thereof:

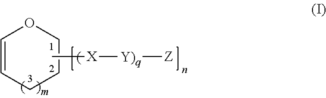

wherein:
m is 0 or 1;
when m is 0, then n is 1 or 2, and when m is 1 then n is 1, 2, or 3;
q is 0 or 1;
each X independently is C₁₋₁₀alkylene, arylene, or C₂₋₁₀polyalkyleneoxide;
each Y independently is O, S, or NR¹;
when q is 0, then each Z independently is C₀₋₁₀alkylene-C₃₋₈cycloalkyl, C₀₋₁₀alkylene-C₃₋₈heterocycloalkyl, C₀₋₁₀alkylene-aryl, C₀₋₁₀alkylene-heteroaryl, C₀₋₁₀alkylene-CN, C₀₋₁₀alkylene-NR¹₃⁺, C₀₋₁₀alkylene-ONO₂, C₀₋₁₀alkylene-OSO₂R², C₀₋₁₀alkylene-OPO₃H₂, C₂₋₁₀polyalkyleneoxide-C₃₋₈cycloalkyl, C₂₋₁₀polyalkyleneoxide-C₃₋₈heterocycloalkyl, C₂₋₁₀polyalkyleneoxide-aryl, C₂₋₁₀polyalkyleneoxide-heteroaryl, C₂₋₁₀polyalkyleneoxide-CN, C₂₋₁₀polyalkyleneoxide-NR¹₃⁺, C₂₋₁₀polyalkyleneoxide-ONO₂, C₂₋₁₀polyalkyleneoxide-OSO₂R², or C₂₋₁₀polyalkyleneoxide-OPO₃H₂;
when q is 1, then each Z independently is C₀₋₁₀alkylene-C₃₋₈cycloalkyl, C₀₋₁₀alkylene-C₃₋₈heterocycloalkyl, C₀₋₁₀alkylene-aryl, C₀₋₁₀alkylene-heteroaryl, C₀₋₁₀alkylene-COOR¹, C₁₋₁₀alkylene-CN, C₁₋₁₀alkylene-OR¹, C₁₋₁₀alkylene-SR¹, C₁₋₁₀alkylene-NR¹₂, C₁₋₁₀alkylene-NR¹₃⁺, C₁₋₁₀alkylene-ONO₂, C₁₋₁₀alkylene-OSO₂R², C₁₋₁₀alkylene-OPO₃H₂, C₂₋₁₀polyalkyleneoxide-C₃₋₈cycloalkyl, C₂₋₁₀polyalkyleneoxide-C₃₋₈heterocycloalkyl, C₂₋₁₀polyalkyleneoxide-aryl, C₂₋₁₀polyalkyleneoxide-heteroaryl, C₂₋₁₀polyalkyleneoxide-COOR¹, C₂₋₁₀polyalkyleneoxide-CN, C₂₋₁₀polyalkyleneoxide-OR¹, C₂₋₁₀polyalkyleneoxide-SR¹, C₂₋₁₀polyalkyleneoxide-NR¹₂, C₂₋₁₀polyalkyleneoxide-NR¹₃⁺, C₂₋₁₀polyalkyleneoxide-ONO₂, C₂₋₁₀polyalkyleneoxide-OSO₂R², C₂₋₁₀polyalkyleneoxide-OPO₃H₂, or

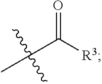

each R¹ independently is H, C₁₋₆alkyl, or aryl;
each R² independently is OR¹ or NR¹₂;
each R³ independently is C₁₋₁₀alkyl, C₀₋₁₀alkylene-C₃₋₈cycloalkyl, C₀₋₁₀alkylene-C₅₋₈heterocycloalkyl [Pro], C₀₋₁₀alkylene-aryl, C₀₋₁₀alkylene-heteroaryl [pyrrole], C₁₋₁₀alkylene-COOR⁴, C₀₋₁₀alkylene-OR⁴, C₀₋₁₀alkylene-SR⁴, C₀₋₁₀alkylene-NR¹₂, C₀₋₁₀alkylene-NR¹₃⁺, C₁₋₁₀alkylene-CN, C₁₋₁₀alkylene-NO₂, C₁₋₁₀alkylene-ONO₂, C₁₋₁₀alkylene-SO₂R², C₁₋₁₀alkylene-OSO₂R², C₁₋₁₀alkylene-PO₃H₂, C₁₋₁₀alkylene-OPO₃H₂, C₂₋₁₀polyalkyleneoxide, C₂₋₁₀polyalkyleneoxide-C₃₋₈cycloalkyl, C₂₋₁₀polyalkyleneoxide-C₅₋₈heterocycloalkyl, C₂₋₁₀polyalkyleneoxide-aryl, C₂₋₁₀polyalkyleneoxide-heteroaryl, C₂₋₁₀polyalkyleneoxide-COOR⁴, C₂₋₁₀polyalkyleneoxide-OR⁴, C₂₋₁₀polyalkyleneoxide-SR⁴, C₂₋₁₀polyalkyleneoxide-NR¹₂, C₂₋₁₀polyalkyleneoxide-NR¹₃⁺, C₂₋₁₀polyalkyleneoxide-CN, C₂₋₁₀polyalkyleneoxide—NO₂, C₂₋₁₀polyalkyleneoxide-SO₃R⁴, C₂₋₁₀polyalkyleneoxide-PO₃H₂, or CH(NR¹₂)R⁵;
each R⁴ independently is H, C₁₋₁₀alkyl, or C₂₋₁₀polyalkylene; and
   each R⁵ independently is H, C₁₋₆alkyl, C₁₋₆alkylene-OR¹, C₁₋₆alkylene-SR¹, C₁₋₆alkylene-COOH, C₁₋₆alkylene-CONR¹₂, C₁₋₆alkylene-NR¹₂, C₁₋₆alkylene-NHC(NH₂)₂, C₁₋₆alkylene-aryl, or C₁₋₆alkylene-heteroaryl.

Yet another aspect of the disclosure provides drug conjugate, or a pharmaceutically acceptable salt thereof, comprising a therapeutic agent and the polymer for Formula (II) or the monomer of Formula (I);
wherein the therapeutic agent and monomer or polymer are attached through a linking group selected from the group consisting of an ester linkage, a thioester linkage, an amide linkage, a carbamate linkage, a carbonate linkage, and a metal ligand bond, and
the linking group is formed from the Z group of the monomer or polymer, and a reactive group on the therapeutic agent, such that:
   (i) Z comprises a hydroxyl group, a thiol group, or an amino group, and the reactive group comprises a carboxylic acid or an activated carboxylic acid; or
   (ii) Z comprises a carboxylic acid or an activated carboxylic acid, and the reactive group comprises a hydroxyl group, a thiol group, or an amino group; or
   (iii) each of Z and the reactive group comprise a metal-binding ligand.
      In some embodiments, the therapeutic agent is an analgesic, anesthetic, antiobiotic, antifungal agent, anticancer agent, antiviral agent, anti-inflammatory agent, or steroid.

Another aspect of the disclosure provides a mucoadhesive formulation comprising a the polymer of Formula (II) and/or a drug conjugate as disclosed herein, and a pharmaceutically acceptable excipient. In some embodiments, the formulation is capable of adhering to a non-keratinized surface in the oral cavity, oropharynx, or both. The formulation can be in the form of a film, ointment, paste, gel, varnish, patch, or spray. In some cases, the formulation further includes one or more additional active ingredients.

Yet another aspect of the disclosure provides a method of treating a disease or condition in a subject. In this method, a non-keratinized surface in the oral cavity, oropharynx, or both of the subject is contacted with the polymer of Formula (II), the drug conjugate described herein, or the formulation described herein to provide a therapeutic beneficial effect on the disease or condition of the subject. In some embodiments, the subject suffers from a disease or condition selected from the group consisting of head and neck cancer, mucositis, periodontitis, gingivitis, necrotizing ulcerative gingivitis, necrotizing ulcerative periodontitis, periocoronitis, candidosis, periodontal abscess, periapical abscess, cellulitis, benign oral cavity tumors, benign oropharyngeal tumors, leukoplakia, and erythroplakia.

Another aspect of the disclosure provides a method of delivering a therapeutic agent to the oral cavity, oropharynx, or both to a subject. In this method, a non-keratinized surface of the oral cavity, oropharynx, or both of the subject is contacted with the drug conjugate and/or the formulation described herein. In some cases, the subject suffers from a disease or condition selected from the group consisting of head and neck cancer, mucositis, periodontitis, candidosis, periodontal abscess, periapical abscess, cellulitis, benign oral cavity tumors, benign oropharyngeal tumors, leukoplakia, and erythroplakia.

Further aspects and advantages will be apparent to those of ordinary skill in the art from a review of the following detailed description, taken in conjunction with the drawings. The description hereafter includes specific embodiments with the understanding that the disclosure is illustrative, and is not intended to limit the invention to the specific embodiments described herein.

### DETAILED DESCRIPTION

Disclosed herein are substituted cyclic ether monomers, polymers, and drug conjugates that are useful for the prevention and treatment of oral diseases and conditions. The compounds disclosed herein, either alone or as part of mucoadhesive formulations, can adhere to non-keratinized surfaces of the oral cavity and/or the oropharynx, such as surfaces that are lined with carbohydrate-based molecules (e.g., glycoproteins). The disclosed compounds and/or mucoadhesive formulations can provide a protective layer over the non-keratinized surfaces. This protective layer can result in pain relief by soothing oral lesions of various etiologies, such as oral mucositis and/or stomatitis that can result from chemotherapy or radiation therapy, irritation due to oral surgery, and traumatic ulcers caused by braces or ill-fitting dentures. When applied to oral surfaces, the compounds disclosed herein (or mucoadhesive formulations thereof) can prevent the spread of pathogens into inflamed and/or diseased tissue.

The substituted cyclic ether polymers disclosed herein are advantageous over previous mucoadhesive systems, which include carbohydrate-based polymer systems. The compounds described herein include carbon-carbon linkages between monomeric units, which are more stable to the harsh chemical conditions of the oral cavity and oropharynx than the oxygen-linked glycosidic bonds of carbohydrates of the previous mucoadhesive systems. The substituted cyclic ether monomers and polymers are further advantageous over traditional carbohydrate-based polymer systems because their structures can be tuned to accommodate bonding to various types of surfaces in the oral cavity and oropharynx by altering their polarity, charge type, charge density, and length. Because diseased tissue is known to have altered properties compared to normal healthy mucosal surfaces, the composition of the substituted cyclic ether polymers described herein can be tailored to bond to the surfaces of diseased, as well as healthy tissue.

The substituted cyclic ether monomers and polymers can be bonded to therapeutic agents to form drug conjugates. The therapeutic agents can be attached to the cyclic ether monomers or polymers through bonds that are capable of cleaving in the oral cavity and/or oropharynx, such as those susceptible to hydrolysis or cleavage by fluoride, thereby enabling the drug conjugates to release and deliver therapeutic agents to the oral cavity and/or oropharynx. The monomers, polymers, and drug conjugates disclosed herein can also be used to form mucoadhesive formulations, such as films, ointments, pastes, gels, varnishes, patches, and sprays that are capable of adhering to and delivering therapeutics to the oral cavity and/or oropharynx.

### Definitions

As used herein, the term “structural unit” is interchangeably referred to as a “monomer,” and refers to a specific cyclic ether moiety of a polymer of Formula (II), as disclosed herein.

As used herein, the term “alkyl” refers to straight chained and branched saturated hydrocarbon groups containing one to thirty carbon atoms, for example, one to twenty carbon atoms, or one to ten carbon atoms. The term Cₙ means the alkyl group has “n” carbon atoms. For example, C₄ alkyl refers to an alkyl group that has 4 carbon atoms. C₁-C₇ alkyl refers to an alkyl group having a number of carbon atoms encompassing the entire range (i.e., 1 to 7 carbon atoms), as well as all subgroups (e.g., 1-6, 2-7, 1-5, 3-6, 1, 2, 3, 4, 5, 6, and 7 carbon atoms). Nonlimiting examples of alkyl groups include, methyl, ethyl, n-propyl, isopropyl, n-butyl, sec-butyl (2-methylpropyl), t-butyl (1,1-dimethylethyl), 3,3-dimethylpentyl, and 2-ethylhexyl. Unless otherwise indicated, an alkyl group can be an unsubstituted alkyl group or a substituted alkyl group.

As used herein, the term “alkylene” refers to an alkyl group having a substituent. For example, the term “alkylene-aryl” refers to an alkyl group substituted with an aryl group. The term Cₙ means the alkylene group has “n” carbon atoms. For example, C₁₋₆ alkylene refers to an alkylene group having a number of carbon atoms encompassing the entire range, as well as all subgroups, as previously described for “alkyl” groups.

As used herein, the term “cycloalkyl” refers to an aliphatic cyclic hydrocarbon group containing three to eight carbon atoms (e.g., 3, 4, 5, 6, 7, or 8 carbon atoms). The term Cₙ means the cycloalkyl group has “n” carbon atoms. For example, C₅ cycloalkyl refers to a cycloalkyl group that has 5 carbon atoms in the ring. C₅-C₈ cycloalkyl refers to cycloalkyl groups having a number of carbon atoms encompassing the entire range (i.e., 5 to 8 carbon atoms), as well as all subgroups (e.g., 5-6, 6-8, 7-8, 5-7, 5, 6, 7, and 8 carbon atoms). Nonlimiting examples of cycloalkyl groups include cyclopropyl, cyclobutyl, cyclopentyl, cyclohexyl, cycloheptyl, and cyclooctyl. Unless otherwise indicated, a cycloalkyl group can be an unsubstituted cycloalkyl group or a substituted cycloalkyl group. Cycloalkyl groups can be saturated or partially unsaturated ring systems optionally substituted with, for example, one to three groups, independently selected alkyl, alkylene-OH, C(O)NH₂, NH₂, oxo (═O), aryl, haloalkyl, halo, and OH.

As used herein, the term “heterocycloalkyl” or “heterocyclic” is defined similarly as cycloalkyl, except the ring contains one to three heteroatoms independently selected from oxygen, nitrogen, or sulfur. Nonlimiting examples of heterocycloalkyl groups include pyrrolidinyl, piperidyl, tetrahydrofuranyl, tetrahydropyranyl, dihydrofuranyl, tetrahydrothienyl, morpholinyl, and the like. Heterocycloalkyl groups can be saturated or partially unsaturated ring systems optionally substituted with, for example, one to three groups, independently selected alkyl, alkyleneOH, C(O)NH₂, NH₂, oxo (═O), aryl, haloalkyl, halo, and OH. Heterocycloalkyl groups optionally can be further N-substituted with alkyl, hydroxyalkyl, alkylene-aryl, and alkylene-heteroaryl. The heterocycloalkyl groups described herein can be isolated, share a carbon atom with another cycloalkyl or heterocycloalkyl group, or fused to another heterocycloalkyl group, a cycloalkyl group, an aryl group and/or a heteroaryl group.

As used herein, “polyalkyleneoxide” refers to a linking moiety having a structure

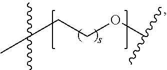

wherein each s is 0, 1, or 2, and the alkyleneoxide chain can be repeated 1, 2, or 3 times. For example, polyalkylene oxide can include a moiety of —CH₂OCH₂CH₂O—, or one, two or three repeating units of ethylene glyocol (CH₂CH₂O)₁₋₃. When “polyalkyleneoxide” is preceded by Cₙ, the Cₙ refers to the total number of carbon atoms in the polyalkyleneoxide chain. For example, C₄polyalkyleneoxide refers to a polyalkyleneoxide chain that has a total of 4 carbon atoms. C₂₋₁₀polyalkyleneoxide refers to a polyalkyleneoxide chain having a total number of carbon atoms encompassed within the entire range (i.e., 2 to 10 carbon atoms), as well as all subgroups (e.g., 1-6, 2-10, 1-5, 3-6, 1, 2, 3, 4, 5, 6, 7, 8, 9, and 10 carbon atoms).

As used herein, the term “aryl” refers to monocyclic or polycyclic (e.g., fused bicyclic and fused tricyclic) carbocyclic aromatic ring systems. Examples of aryl groups include, but are not limited to, phenyl, naphthyl, tetrahydronaphthyl, phenanthrenyl, biphenylenyl, indanyl, indenyl, anthracenyl, and fluorenyl. Unless otherwise indicated, an aryl group can be an unsubstituted aryl group or a substituted aryl group.

As used herein, the term “arylene” refers to an aryl group having a substituent. For example, the term “arylene-OH” refers to an aryl group substituted with a hydroxyl group.

As used herein, the term “heteroaryl” refers to monocyclic or polycyclic (e.g., fused bicyclic and fused tricyclic) aromatic ring systems, wherein one to four-ring atoms are selected from oxygen, nitrogen, or sulfur, and the remaining ring atoms are carbon, said ring system being joined to the remainder of the molecule by any of the ring atoms. Nonlimiting examples of heteroaryl groups include, but are not limited to, pyridyl, pyridazinyl, pyrazinyl, pyrimidinyl, pyrrolyl, pyrazolyl, imidazolyl, thiazolyl, tetrazolyl, oxazolyl, isooxazolyl, thiadiazolyl, oxadiazolyl, furanyl, thiophenyl, quinolinyl, isoquinolinyl, benzoxazolyl, benzimidazolyl, benzothiazolyl, triazinyl, triazolyl, purinyl, pyrazinyl, purinyl, indolinyl, phthalzinyl, indazolyl, quinolinyl, isoquinolinyl, cinnolinyl, quinazolinyl, naphthyridinyl, pyridopyridinyl, indolyl, 3H-indolyl, pteridinyl, quinooxalinyl, 1,3-dioxaindanyl, anisothiazolyl, benzofuranyl, isobenzofuryl, benzothienyl, isoindolyl, chromanyl, benzoimidazolyl, benzoxazolyl, pyranyl, furazanyl, dihydrobenzofuryl, dihydroisobenzofuryl, dihydroquinolyl, dihydroisoquinolyl, dihydrobenzoxazolyl, dihydropteridinyl, benzoxazolyl, benzisoxazolyl, benzodioxazolyl, and benzotriazolyl. Unless otherwise indicated, a heteroaryl group can be an unsubstituted heteroaryl group or a substituted heteroaryl group.

As used herein, the term “metal-binding ligand” refers to Lewis base that can complex to a metal cation through an electron lone pair. Examples of metal-binding ligands include solvents (e.g., pyridine, ethanol, methanol, trifluoroethanol, acetonitrile, dimethylsulfoxide, dimethyl sulfide, diethylether, or tetrahydrofuran) and halides (e.g., F⁻, Cl⁻, Br⁻, or I⁻).

A used herein, the term “substituted,” when used to modify a chemical functional group, refers to the replacement of at least one hydrogen radical on the functional group with a substituent. Substituents can include, but are not limited to, alkyl, cycloalkyl, alkenyl, cycloalkenyl, alkynyl, heterocycloalkyl, thioether, polythioether, aryl, heteroaryl, hydroxyl, oxy, alkoxy, heteroalkoxy, aryloxy, heteroaryloxy, ester, thioester, carboxy, cyano, nitro, amino, amido, acetamide, and halo (e.g., fluoro, chloro, bromo, or iodo). When a chemical functional group includes more than one substituent, the substituents can be bound to the same carbon atom or to two or more different carbon atoms. A substituted chemical functional group can itself include one or more substituents.

It will be appreciated by those skilled in the art that compounds of the disclosure having a chiral center may exist in and be isolated in optically active and racemic forms. Some compounds may exhibit polymorphism. It is to be understood that the present disclosure encompasses any racemic, optically-active, polymorphic, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase. When a bond in a compound formula herein is drawn in a non-stereochemical manner (e.g. flat), the atom to which the bond is attached includes all stereochemical possibilities.

As used herein, “therapeutic agent” refers to any compound that can ameliorate, attenuate, or eliminate one or more symptoms of a particular disease or condition, or prevents or delays the onset of one of more symptoms of a particular disease or condition.

As used herein, the term “therapeutically effective amount” means an amount of a compound or combination of therapeutically active compounds that ameliorates, attenuates or eliminates one or more symptoms of a particular disease or condition, or prevents or delays the onset of one of more symptoms of a particular disease or condition.

As used herein, the terms “patient” and “subject” may be used interchangeably and mean animals, such as dogs, cats, cows, horses, and sheep (i.e., non-human animals) and humans. Particular patients or subjects are mammals (e.g., humans). The terms patient and subject includes males and females.

As used herein, the term “pharmaceutically acceptable” means that the referenced substance, such as a compound of the present disclosure, or a formulation containing the compound, or a particular excipient, are safe and suitable for administration to a patient or subject.

As used herein the terms “treating”, “treat” or “treatment” and the like include preventative (e.g., prophylactic) and palliative treatment. In some cases, the treating refers to treating a symptom of a disorder or disease as disclosed herein.

As used herein, the term “mucoadhesive” refers to the ability to adhere to a mucosal surface.

As used herein, the term “film” refers to a continuous layer of polymer.

As used herein, the term “mucoadhesive film” refers to a film that can adhere to a mucosal surface.

As used herein, the term “guided tissue regeneration film” refers to a film that is used as a barrier to promote the growth of new bone and tissue.

As used herein, the term “ointment” refers to an oily preparation that is applied to the surfaces of the oral cavity and/or oropharynx for medicinal purposes.

As used herein, the term “paste” refers to an ointment in which a powder is suspended.

As used herein, the term “gel” refers to a semi-solid emulsion that is applied to the surfaces of the oral cavity and/or oropharynx for medicinal purposes.

As used herein, the term “varnish” refers to a coating that is applied to the teeth and that can become hard, e.g., upon contact with saliva.

As used herein, the term “patch” refers to a device that includes a non-impermeable backing (e.g., cellulose) upon which materials can be layered.

As used herein, the term “spray” refers to a liquid medication that can be atomized and applied to surfaces of the oral cavity and/or oropharynx for medicinal purposes.

As used herein, the term “oral cavity” refers to the part of the mouth behind the gums and teeth that is bounded above by the hard and soft palates and below by the tongue and by the mucous membrane connecting it with the inner part of the mandible.

As used herein, the term “oropharynx” refers to the part of the mouth that includes the back one-third of the tongue, the soft palate, the side and back walls of the throat, and the tonsils.

As used herein, the phrase “capable of adhering to a surface” refers to the ability of an object to stay attached to a surface for a period of time. When referring to a formulation that is capable of adhering to a surface in the oral cavity and/or oropharynx, the phrase means that the formulation stays attached to a surface in the oral cavity and/or oropharynx for a period of time. In some cases, that period of time is, e.g., 1 hour to 21 days or more (30 days, 6 weeks, 2 months, 3 months, 4 months, or 6 months).

### Substituted Cyclic Ether Monomers

One aspect of the disclosure provides monomers of Formula (I), or pharmaceutically acceptable salts thereof:

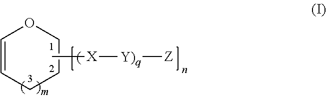

wherein:
m is 0 or 1;
when m is 0, then n is 1 or 2, and when m is 1 then n is 1, 2, or 3;
q is 0 or 1;
each X independently is C₁₋₁₀alkylene, arylene, or C₂₋₁₀polyalkyleneoxide;
each Y independently is O, S, or NR¹;
when q is 0, then each Z independently is C₀₋₁₀alkylene-C₃₋₈cycloalkyl, C₀₋₁₀alkylene-C₃₋₈heterocycloalkyl, C₀₋₁₀alkylene-aryl, C₀₋₁₀alkylene-heteroaryl, C₀₋₁₀alkylene-CN, C₀₋₁₀alkylene-NR¹₃⁺, C₀₋₁₀alkylene-ONO₂, C₀₋₁₀alkylene-OSO₂R², C₀₋₁₀alkylene-OPO₃H₂, C₂₋₁₀polyalkyleneoxide-C₃₋₈cycloalkyl, C₂₋₁₀polyalkyleneoxide-C₃₋₈heterocycloalkyl, C₂₋₁₀polyalkyleneoxide-aryl, C₂₋₁₀polyalkyleneoxide-heteroaryl, C₂₋₁₀polyalkyleneoxide-CN, C₂₋₁₀polyalkyleneoxide-NR¹₃⁺, C₂₋₁₀polyalkyleneoxide-ONO₂, C₂₋₁₀polyalkyleneoxide-OSO₂R², or C₂₋₁₀polyalkyleneoxide-OPO₃H₂;
when q is 1, then each Z independently is C₀₋₁₀alkylene-C₃₋₈cycloalkyl, C₀₋₁₀alkylene-C₃₋₈heterocycloalkyl, C₀₋₁₀alkylene-aryl, C₀₋₁₀alkylene-heteroaryl, C₀₋₁₀alkylene-COOR¹, C₁₋₁₀alkylene-CN, C₁₋₁₀alkylene-OR¹, C₁₋₁₀alkylene-SR¹, C₁₋₁₀alkylene-NR¹₂, C₁₋₁₀alkylene-NR¹₃⁺, C₁₋₁₀alkylene-ONO₂, C₁₋₁₀alkylene-OSO₂R², C₁₋₁₀alkylene-OPO₃H₂, C₂₋₁₀polyalkyleneoxide-C₃₋₈cycloalkyl, C₂₋₁₀polyalkyleneoxide-C₃₋₈heterocycloalkyl, C₂₋₁₀polyalkyleneoxide-aryl, C₂₋₁₀polyalkyleneoxide-heteroaryl, C₂₋₁₀polyalkyleneoxide-COOR¹, C₂₋₁₀polyalkyleneoxide-CN, C₂₋₁₀polyalkyleneoxide-OR¹, C₂₋₁₀polyalkyleneoxide-SR¹, C₂₋₁₀polyalkyleneoxide-NR¹₂, C₂₋₁₀polyalkyleneoxide-NR¹₃⁺, C₂₋₁₀polyalkyleneoxide-ONO₂, C₂₋₁₀polyalkyleneoxide-OSO₂R², C₂₋₁₀polyalkyleneoxide-OPO₃H₂, or

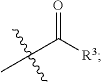

each R¹ independently is H, C₁₋₆alkyl, or aryl;
each R² independently is OR¹ or NR¹₂;
each R³ independently is C₁₋₁₀alkyl, C₀₋₁₀alkylene-C₃₋₈cycloalkyl, C₀₋₁₀alkylene-C₅₋₈heterocycloalkyl, C₀₋₁₀alkylene-aryl, C₀₋₁₀alkylene-heteroaryl, C₁₋₁₀alkylene-COOR⁴, C₀₋₁₀alkylene-OR⁴, C₀₋₁₀alkylene-SR⁴, C₀₋₁₀alkylene-NR¹₂, C₀₋₁₀alkylene-NR¹₃⁺, C₁₋₁₀alkylene-CN, C₁₋₁₀alkylene-NO₂, C₁₋₁₀alkylene-ONO₂, C₁₋₁₀alkylene-SO₂R², C₁₋₁₀alkylene-OSO₂R², C₁₋₁₀alkylene-PO₃H₂, C₁₋₁₀alkylene-OPO₃H₂, C₂₋₁₀polyalkyleneoxide, C₂₋₁₀polyalkyleneoxide-C₃₋₈cycloalkyl, C₂₋₁₀polyalkyleneoxide-C₅₋₈heterocycloalkyl, C₂₋₁₀polyalkyleneoxide-aryl, C₂₋₁₀polyalkyleneoxide-heteroaryl, C₂₋₁₀polyalkyleneoxide-COOR⁴, C₂₋₁₀polyalkyleneoxide-OR⁴, C₂₋₁₀polyalkyleneoxide-SR⁴, C₂₋₁₀polyalkyleneoxide-NR¹₂, C₂₋₁₀polyalkyleneoxide-NR¹₃⁺, C₂₋₁₀polyalkyleneoxide-CN, C₂₋₁₀polyalkyleneoxide—NO₂, C₂₋₁₀polyalkyleneoxide-SO₃R⁴, C₂₋₁₀polyalkyleneoxide-PO₃H₂, or CH(NR¹₂)R⁵ [primary amino acids].
each R⁴ independently is H, C₁₋₁₀alkyl, or C₂₋₁₀polyalkylene; and
each R⁵ independently is H, C₁₋₆alkyl, C₁₋₆alkylene-OR¹, C₁₋₆alkylene-SR¹, C₁₋₆alkylene-COOH, C₁₋₆alkylene-CONR¹₂, C₁₋₆alkylene-NR¹₂, C₁₋₆alkylene-NHC(NH₂)₂, C₁₋₆alkylene-aryl, C₁₋₆alkylene-heteroaryl.

In some embodiments, the compound of Formula (I) is complexed to a metal cation through at least one heteroatom (e.g., N, O, or S) of the monomer. The metal cation can be a metal cation, in any oxidation state, capable of complexing to the heteroatom. In some cases, the metal cation is an f-block metal, such as a lanthanide. Suitable lanthanides include lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), and lutetium (Lu). For example, the metal cation can be cerium or lanthanum. In some cases, the metal cation can be a d-block metal. Suitable d-block cations include scandium (Sc), titanium (Ti), vanadium (V), chromium (Cr), manganese (Mn), iron (Fe), cobalt (Co), nickel (Ni), copper (Cu), zinc (Zn), yttrium (Y), zirconium (Zr), niobium (Nb), molybdenum (Mo), technetium (Tc), ruthenium (Ru), rhodium (Rh), palladium (Pd), silver (Ag), cadmium (Cd), hafnium (Hf), tantalum (Ta), tungsten (W), rhenium (Re), osmium (Os), iridium (Ir), platinum (Pt), gold (Au), and mercury (Hg). For example, the metal cation can be zinc and silver. In various embodiments, the metal cation can be a p-block metal. Suitable p-block metals include aluminum (Al), gallium (Ga), germanium (Ge), indium (In), tin (Sn), antimony (Sb), thallium (Tl), lead (Pb), bismuth (Bi), and polonium (Po). For example, the metal cation can be tin. In some cases, the metal cation can be an alkaline earth metal. Suitable alkaline earth metals include beryllium (Be), magnesium (Mg), calcium (Ca), strontium (Sr), barium (Ba), and radium (Ra). For example, the metal cation can be magnesium or calcium. In some embodiments, the metal cation is selected from the group consisting of lanthanum, cerium, tin, silver, zinc, magnesium, and calcium.

In any of the embodiments disclosed herein wherein Z includes cycloalkyl, Z can include, for example, cyclopentyl or cyclohexyl. In any the embodiments disclosed herein wherein Z includes heterocycloalkyl, Z can include, for example, pyrrolidinyl, pyrrolidonyl, piperidyl, tetrahydrofuranyl, tetrahydropyranyl, dihydrofuranyl, tetrahydrothienyl, or morpholinyl. In any of the embodiments disclosed herein wherein Z includes aryl, Z can include, for example, phenyl, naphthyl, tetrahydronaphthyl, phenanthrenyl, biphenylenyl, indanyl, indenyl, anthracenyl, or fluorenyl. In any of the embodiments disclosed herein wherein Z includes heteroaryl, then Z can include, for example, pyridyl, pyrazinyl, pyrimidinyl, pyrrolyl, pyrazolyl, imidazolyl, thiazolyl, tetrazolyl, oxazolyl, isooxazolyl, thiadiazolyl, oxadiazolyl, furanyl, thiophenyl, quinolinyl, isoquinolinyl, benzoxazolyl, benzimidazolyl, or benzothiazolyl.

In some cases, q is 0. In some of these cases, Z is C₀₋₁₀alkylene-CN, C₀₋₁₀alkylene-NR¹₃⁺, C₀₋₁₀alkylene-ONO₂, C₀₋₁₀alkylene-OSO₂R₂, or C₀₋₁₀alkylene-OPO₃H₂, R¹ is H, methyl, ethyl, n-propyl, isopropyl, n-butyl, s-butyl, t-butyl, pentyl, or hexyl, and R² is OH, OMe, or NMe₂. For example, Z can be —CH₂NR¹₃⁺ or —CH₂CH₂NR¹₃⁺ and each R¹ can methyl, ethyl, propyl, butyl, pentyl, or hexyl, such as —CH₂NCH₃⁺. In some embodiments, Z can be —OSO₂R², —CH₂OSO₂R², or —CH₂CH₂OSO₂R², such as —OSO₂H, —CH₂OSO₂H, or —CH₂CH₂OSO₂H, —OSO₂NMe₂, —CH₂OSO₂NMe₂, or —CH₂CH₂OSO₂NMe₂. In various embodiments, Z is C₀₋₁₀alkylene-C₃₋₈cycloalkyl (e.g., -cyclopentyl, —CH₂cyclopentyl, —CH₂CH₂cyclopentyl, -cyclohexyl, —CH₂cyclohexyl, or —CH₂CH₂cyclohexyl), C₀₋₁₀alkylene-C₃₋₈heterocycloalkyl (e.g., -pyrrolidinyl, —CH₂pyrrolidinyl, —CH₂CH₂pyrrolidinyl, -pyrrolidonyl, —CH₂pyrrolidonyl, or —CH₂CH₂pyrrolidonyl), C₀₋₁₀alkylene-aryl (e.g., -Ph, —CH₂Ph, —CH₂CH₂Ph), or C₀₋₁₀alkylene-heteroaryl (e.g., -imdazolyl, —CH₂imdazolyl, —CH₂CH₂imdazolyl, -pyrrolyl, —CH₂pyrrolyl, —CH₂CH₂pyrrolyl, -thiophenyl, —CH₂thiophenyl, or —CH₂CH₂thiophenyl). In some cases, Z is

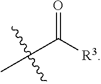

In some of these embodiments, R³ is C₁₋₁₀alkyl (e.g., methyl, ethyl, n-propyl, isopropyl, n-butyl, s-butyl, t-butyl, pentyl, or hexyl, heptyl, octyl, nonyl, or decyl), C₀₋₁₀alkylene-C₃₋₈cycloalkyl (e.g., -cyclopentyl, —CH₂cyclopentyl, —CH₂CH₂cyclopentyl, -cyclohexyl, —CH₂cyclohexyl, or —CH₂CH₂cyclohexyl), C₀₋₁₀alkylene-C₃₋₈heterocycloalkyl (e.g., -pyrrolidinyl, —CH₂pyrrolidinyl, —CH₂CH₂pyrrolidinyl, -pyrrolidonyl, —CH₂pyrrolidonyl, or —CH₂CH₂pyrrolidonyl), C₀₋₁₀alkylene-aryl (e.g., -Ph, —CH₂Ph, or —CH₂CH₂Ph), C₀₋₁₀alkylene-heteroaryl (e.g., -imdazolyl, —CH₂imdazolyl, —CH₂CH₂imdazolyl, -pyrrolyl, —CH₂pyrrolyl, —CH₂CH₂pyrrolyl, -thiophenyl, —CH₂thiophenyl, or —CH₂CH₂thiophenyl), C₂₋₁₀polyalkyleneoxide, C₂₋₁₀polyalkyleneoxide-C₃₋₈cycloalkyl, C₂₋₁₀polyalkyleneoxide-C₅₋₈heterocycloalkyl, C₂₋₁₀polyalkyleneoxide-aryl, or C₂₋₁₀polyalkyleneoxide-heteroaryl. In some embodiments, Z can be

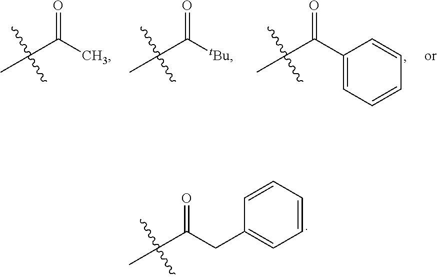

In various cases, q is 1. In some of these cases, X is C₁₋₁₀alkylene (e.g., methylene, ethylene, propylene, butylene, pentylene, hexylene, heptylene, octylene, nonylene, or decylene), such as C₁₋₆alkylene or C₁₋₄alkylene. For example X can be —CH₂− or −CH₂CH₂−. In some cases, X is arylene (e.g., phenylene, naphthylene, tetrahydronaphthylene, phenanthrenylene, biphenylenylene, indanylene, indenylene, anthracenylene, and fluorenylene). For example, X can be phenylene, naphthylene, or anthracenylene. In various embodiments, X is C₂₋₁₀polyalkyleneoxide, such as C₂₋₆polyalkyleneoxide or C₂₋₄polyalkyleneoxide. In some cases, the polyalkyleneoxide is polyethyleneoxide, polypropyleneoxide, or polybutyleneoxide.

In some embodiments, Y is O. In various embodiments, Y is S. In some cases, Y is NR¹. In some embodiments, R¹ is H. In some cases, R¹ is C₁₋₆alkyl (e.g., methyl, ethyl, propyl, butyl, pentyl, or hexyl). For example, Y can be NMe, NEt, or NPr.

In some cases, Z is C₀₋₁₀alkylene-ONO₂ (e.g., CH₂ONO₂ or CH₂CH₂ONO₂), C₀₋₁₀alkylene-OSO₂R² (e.g., CH₂OSO₂R² or CH₂CH₂OSO₂R²), or C₀₋₁₀alkylene-OPO₃H₂ (e.g., CH₂OPO₃H₂ or CH₂CH₂OPO₃H₂). In some of these embodiments, R² is OR¹, and R¹ is H, methyl, ethyl, n-propyl, isopropyl, n-butyl, s-butyl, or t-butyl (e.g., OH, OMe, OEt, OPr, OⁱPr, OⁿBu, O^{s}Bu, or O^{t}Bu). For example, Z can be —CH₂OSO₃H, —CH₂OSO₃Me, —CH₂OSO₃Et, and —CH₂OSO₃^{t}Bu. In some cases, R² is NR¹₂, and each R¹ independently is H, methyl, or phenyl (e.g., NH₂, NHMe, NMe₂, NHPh, and NMePh). For example, Z can be —CH₂OSO₂NH₂, —CH₂OSO₂NHMe, —CH₂OSO₂NMe₂, —CH₂OSO₂NHPh, and —CH₂OSO₂NMePh. In some cases, Z is C₀₋₁₀alkylene-C₃₋₈cycloalkyl (e.g., -cyclopentyl, —CH₂cyclopentyl, —CH₂CH₂cyclopentyl, -cyclohexyl, —CH₂cyclohexyl, or —CH₂CH₂cyclohexyl), C₀₋₁₀alkylene-C₅₋₈heterocycloalkyl (e.g., -pyrrolidinyl, —CH₂pyrrolidinyl, —CH₂CH₂pyrrolidinyl, -pyrrolidonyl, —CH₂pyrrolidonyl, or —CH₂CH₂pyrrolidonyl), C₀₋₁₀alkylene-aryl (e.g., -Ph, —CH₂Ph, or —CH₂CH₂Ph), or C₀₋₁₀alkylene-heteroaryl (e.g., -imdazolyl, —CH₂imdazolyl, —CH₂CH₂imdazolyl, -pyrrolyl, —CH₂pyrrolyl, —CH₂CH₂pyrrolyl, -thiophenyl, —CH₂thiophenyl, or —CH₂CH₂thiophenyl). In various embodiments, Z is —CH₂Ph,

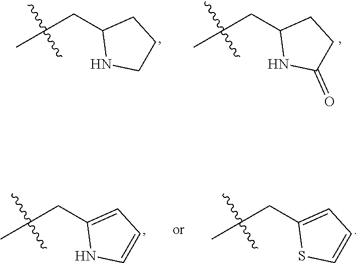 [SMILES: *CC1CCCN1 ; *CC1CCC(=O)N1 ; *Cc1ccc[nH]1 ; *Cc1cccs1]

In some cases, Z is C₀₋₁₀alkylene-COOR¹, C₀₋₁₀alkylene-CN, C₁₋₁₀alkylene-OR¹, C₁₋₁₀alkylene-SR¹, C₁₋₁₀alkylene-NR¹₂, or C₁₋₁₀alkylene-NR¹₃⁺, and R¹ can be H, methyl, ethyl, n-propyl, isopropyl, n-butyl, s-butyl, or t-butyl. For example, Z can be —CH₂OH, —CH₂NH₂, —CH₂N(CH₃)₃⁺, or —CH₂COOH. In some cases, Z is

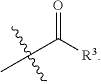

In some of these embodiments, R³ is C₁₋₁₀alkyl, C₀₋₁₀alkylene-C₃₋₈cycloalkyl (e.g., -cyclopentyl, —CH₂cyclopentyl, —CH₂CH₂cyclopentyl, -cyclohexyl, —CH₂cyclohexyl, or —CH₂CH₂cyclohexyl), C₀₋₁₀alkylene-C₅₋₈heterocycloalkyl (e.g., -pyrrolidinyl, —CH₂pyrrolidinyl, —CH₂CH₂pyrrolidinyl, -pyrrolidonyl, —CH₂pyrrolidonyl, —CH₂CH₂pyrrolidonyl)), C₀₋₁₀alkylene-aryl (e.g., -Ph, —CH₂Ph, or —CH₂CH₂Ph), C₀₋₁₀alkylene-heteroaryl (e.g., -imdazolyl, —CH₂imdazolyl, —CH₂CH₂imdazolyl, -pyrrolyl, —CH₂pyrrolyl, —CH₂CH₂pyrrolyl, -thiophenyl, —CH₂thiophenyl, —CH₂CH₂thiophenyl), C₂₋₁₀polyalkyleneoxide, C₂₋₁₀polyalkyleneoxide-C₃₋₈cycloalkyl, C₂₋₁₀polyalkyleneoxide-C₅₋₈heterocycloalkyl, C₂₋₁₀polyalkyleneoxide-aryl, or C₂₋₁₀polyalkyleneoxide-heteroaryl). For example, Z can be

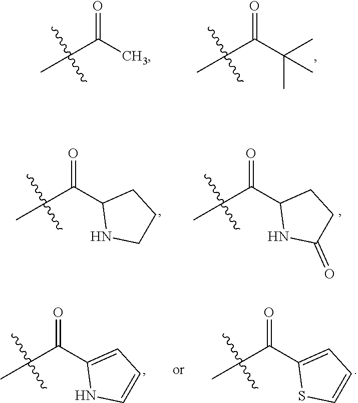

In some cases, R³ is C₁₋₁₀alkylene-COOR⁴, C₀₋₁₀alkylene-OR⁴, C₀₋₁₀alkylene-SR⁴, C₀₋₁₀alkylene-NR¹₂, C₀₋₁₀alkylene-NR¹₃⁺, C₁₋₁₀alkylene-CN, C₁₋₁₀alkylene-NO₂, C₁₋₁₀alkylene-ONO₂, C₁₋₁₀alkylene-SO₂R², C₁₋₁₀alkylene-OSO₂R², C₀₋₁₀alkylene-PO₃H₂, C₀₋₁₀alkylene-OPO₃H₂, C₂₋₁₀polyalkyleneoxide-COOR⁴, C₂₋₁₀polyalkyleneoxide-OR⁴, C₂₋₁₀polyalkyleneoxide-SR⁴, C₂₋₁₀polyalkyleneoxide-NR¹₂, C₂₋₁₀polyalkyleneoxide-NR¹₃⁺, C₂₋₁₀polyalkyleneoxide-CN, C₂₋₁₀polyalkyleneoxide—NO₂, C₂₋₁₀polyalkyleneoxide-SO₃R⁴, or C₂₋₁₀polyalkyleneoxide-PO₃H₂. For example, R³ can be —CH₂N(CH₃)₃⁺ or —CH₂CH₂N(CH₃)₃⁺. In some cases, R³ is CH(NR¹₂)R⁵. In some of these embodiments, R⁵ is H, C₁₋₆alkyl, C₁₋₆alkylene-aryl, or C₁₋₆alkylene-heteroaryl. For example, R⁵ can include —H, —CH₃, —CH(CH₃)₂, —CH₂CH(CH₃)₂, —CH(CH₃)(CH₂CH₃), —CH₂Ph, —CH₂Ph(OH). In various embodiments, R⁵ is C₁₋₆alkylene-OR¹, C₁₋₆alkylene-SR¹, C₁₋₆alkylene-COOH, C₁₋₆alkylene-CONR¹₂, C₁₋₆alkylene-NR¹₂, or C₁₋₆alkylene-NHC(NH₂)₂. For example, R⁵ is —CH₂OH, —CH(CH₃)OH, —CH₂SH, —CH₂CH₂SCH₃, —CH₂COOH, —CH₂CH₂COOH, —CH₂CONH₂, —CH₂CH₂CONH₂, —(CH₂)₄NH₂, or —(CH₂)₃NHC(NH₂)NH₂⁺.

In some embodiments, the cyclic ether monomer is selected from the group consisting of:

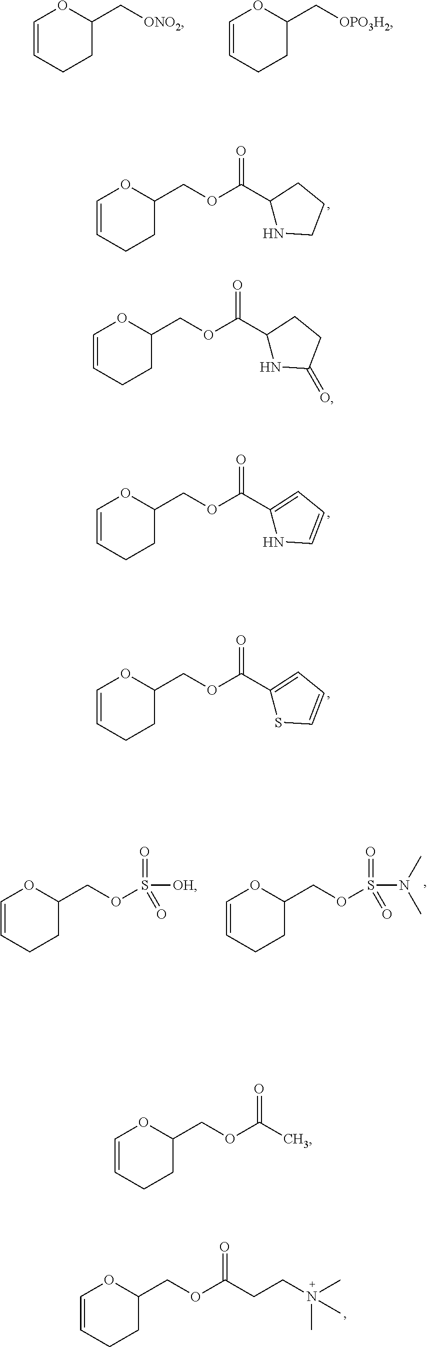

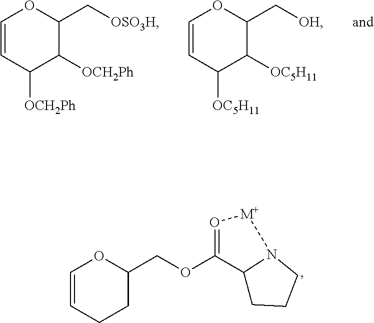

wherein M⁺ is a metal cation as described herein, such as Zn²⁺, Sn²⁺, Sn⁴⁺, or Ag⁺. The metal cation can be further complexed to one or more metal binding ligands. Suitable metal binding ligands include, for example, solvents (e.g., pyridine, ethanol, methanol, trifluoroethanol, acetonitrile, dimethylsulfoxide, dimethyl sulfide, diethylether, or tetrahydrofuran) or halides (e.g., F⁻, Cl⁻, Br⁻, or I⁻). For example, when M is Zn²⁺, it can be further complexed to two Cl⁻ moieties, and when M is Ag⁺, it can be further complexed to pyridine.

### Substituted Cyclic Ether Polymers

Another aspect of the disclosure provides substituted cyclic ether polymers, or pharmaceutically acceptable salts or solvates thereof. The polymers disclosed herein are advantageous because they exhibit a flexible cyclic ether backbone with a range of potential pendant functional groups. These pendant functional groups can provide the polymer with biological function, such as therapeutic activity and/or adherence to the oral cavity and/or oropharynx.

In particular, disclosed herein are polymers having a structure of Formula (II), or pharmaceutically acceptable salts or solvates thereof:

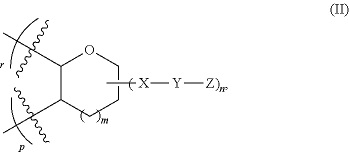

wherein
p+r is 2 to 500;
m is 0 or 1;
when m is 0, then n is 1 or 2, and when m is 1, then n is 1, 2, or 3;
each q is 0 or 1;
each X independently is C₁₋₁₀alkylene, arylene, C₂₋₁₀polyalkyleneoxide, or absent;
each Y independently is O, S, or NR¹;
when q is 0, then each Z independently is C₁₋₁₀alkyl, C₀₋₁₀alkylene-C₃₋₈cycloalkyl, C₀₋₁₀alkylene-C₃₋₈heterocycloalkyl, C₀₋₁₀alkylene-aryl, C₀₋₁₀alkylene-heteroaryl, C₀₋₁₀alkylene-COOR¹, C₀₋₁₀alkylene-CN, C₀₋₁₀alkylene-NR¹₃⁺, C₀₋₁₀alkylene-ONO₂, C₀₋₁₀alkylene-OSO₂R², C₀₋₁₀alkylene-OPO₃H₂, C₂₋₁₀polyalkyleneoxide, C₂₋₁₀polyalkyleneoxide-C₃₋₈cycloalkyl, C₂₋₁₀polyalkyleneoxide-C₃₋₈heterocycloalkyl, C₂₋₁₀polyalkyleneoxide-aryl, C₂₋₁₀polyalkyleneoxide-heteroaryl, C₂₋₁₀polyalkyleneoxide-COOR¹, C₂₋₁₀polyalkyleneoxide-CN, C₂₋₁₀polyalkyleneoxide-NR¹₂, C₂₋₁₀polyalkyleneoxide-NR¹₃⁺, C₂₋₁₀polyalkyleneoxide-ONO₂, C₂₋₁₀polyalkyleneoxide-OSO₂R², C₂₋₁₀polyalkyleneoxide-OPO₃H₂, or

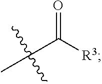

when q is 1, then each Z independently is H, C₁₋₁₀alkyl, C₀₋₁₀alkylene-C₃₋₈cycloalkyl, C₀₋₁₀alkylene-C₃₋₈heterocycloalkyl, C₀₋₁₀alkylene-aryl, C₀₋₁₀alkylene-heteroaryl, C₀₋₁₀alkylene-COOR¹, C₁₋₁₀alkylene-CN, C₁₋₁₀alkylene-OR¹, C₁₋₁₀alkylene-SR¹, C₁₋₁₀alkylene-NR¹₂, C₁₋₁₀alkylene-NR¹₃⁺, C₁₋₁₀alkylene-ONO₂, C₁₋₁₀alkylene-OSO₂R², C₁₋₁₀alkylene-OPO₃H₂, C₂₋₁₀polyalkyleneoxide, C₂₋₁₀polyalkyleneoxide-C₃₋₈cycloalkyl, C₂₋₁₀polyalkyleneoxide-C₃₋₈heterocycloalkyl, C₂₋₁₀polyalkyleneoxide-aryl, C₂₋₁₀polyalkyleneoxide-heteroaryl, C₂₋₁₀polyalkyleneoxide-COOR¹, C₂₋₁₀polyalkyleneoxide-CN, C₂₋₁₀polyalkyleneoxide-OR¹, C₂₋₁₀polyalkyleneoxide-SR¹, C₂₋₁₀polyalkyleneoxide-NR¹₂, C₂₋₁₀polyalkyleneoxide-NR¹₃⁺, C₂₋₁₀polyalkyleneoxide-ONO₂, C₂₋₁₀polyalkyleneoxide-OSO₂R², C₂₋₁₀polyalkyleneoxide-OPO₃H₂, or

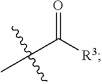

each R¹ independently is H, C₁₋₆alkyl, or aryl;
each R² independently is OR¹ or NR¹₂;
each R³ independently is C₁₋₁₀alkyl, C₀₋₁₀alkylene-C₃₋₈cycloalkyl, C₀₋₁₀alkylene-C₅₋₈heterocycloalkyl, C₀₋₁₀alkylene-aryl, C₀₋₁₀alkylene-heteroaryl, C₁₋₁₀alkylene-COOR⁴, C₀₋₁₀alkylene-OR⁴, C₀₋₁₀alkylene-SR⁴, C₀₋₁₀alkylene-NR¹₂, C₀₋₁₀alkylene-NR¹₃⁺, C₁₋₁₀alkylene-CN, C₁₋₁₀alkylene-NO₂, C₁₋₁₀alkylene-ONO₂, C₁₋₁₀alkylene-SO₂R², C₁₋₁₀alkylene-OSO₂R², C₀₋₁₀alkylene-PO₃H₂, C₀₋₁₀alkylene-OPO₃H₂, C₂₋₁₀polyalkyleneoxide, C₂₋₁₀polyalkyleneoxide-C₃₋₈cycloalkyl, C₂₋₁₀polyalkyleneoxide-C₅₋₈heterocycloalkyl, C₂₋₁₀polyalkyleneoxide-aryl, C₂₋₁₀polyalkyleneoxide-heteroaryl, C₂₋₁₀polyalkyleneoxide-COOR⁴, C₂₋₁₀polyalkyleneoxide-OR⁴, C₂₋₁₀polyalkyleneoxide-SR⁴, C₂₋₁₀polyalkyleneoxide-NR¹₂, C₂₋₁₀polyalkyleneoxide-NR¹₃⁺, C₂₋₁₀polyalkyleneoxide-CN, C₂₋₁₀polyalkyleneoxide—NO₂, C₂₋₁₀polyalkyleneoxide-SO₃R⁴, C₂₋₁₀polyalkyleneoxide-PO₃H₂, or CH(NR¹₂)R⁵;
each R⁴ independently is H, C₁₋₁₀alkyl, or C₂₋₁₀polyalkyleneoxide; and
each R⁵ independently is H, C₁₋₆alkyl, C₁₋₆alkylene-OR¹, C₁₋₆alkylene-SR¹, C₁₋₆alkylene-COOH, C₁₋₆alkylene-CONR¹₂, C₁₋₆alkylene-NR¹₂, C₁₋₆alkylene-NHC(NH₂)₂, C₁₋₆alkylene-aryl, and C₁₋₆alkylene-heteroaryl.

The monomers that make of the polymer of Formula (II) need not be the same monomers, i.e., the options for m, n, q, X, Y, and Z need not be the same for each monomer of the polymer.

In some embodiments, the polymer comprises a metal cation complexed to at least one heteroatom (e.g., N, O, or S) of the polymer. The metal cation can be any metal cation capable of complexing to the heteroatom, as described in the monomer section, supra. In some cases, the metal cation can be magnesium or calcium. In some embodiments, the metal cation is selected from the group consisting of lanthanum, cerium, tin, silver, zinc, magnesium, and calcium.

In any of the embodiments disclosed herein wherein Z includes cycloalkyl, Z can include, for example, cyclopentyl or cyclohexyl. In any of the embodiments disclosed herein wherein Z includes heterocycloalkyl, Z can include, for example, pyrrolidinyl, pyrrolidonyl, piperidyl, tetrahydrofuranyl, tetrahydropyranyl, dihydrofuranyl, tetrahydrothienyl, or morpholinyl. In any of the embodiments disclosed herein wherein Z includes aryl, Z can include, for example, phenyl, naphthyl, tetrahydronaphthyl, phenanthrenyl, biphenylenyl, indanyl, indenyl, anthracenyl, or fluorenyl. In any of the embodiments disclosed herein wherein Z includes heteroaryl, then Z can include, for example, pyridyl, pyrazinyl, pyrimidinyl, pyrrolyl, pyrazolyl, imidazolyl, thiazolyl, tetrazolyl, oxazolyl, isooxazolyl, thiadiazolyl, oxadiazolyl, furanyl, thiophenyl, quinolinyl, isoquinolinyl, benzoxazolyl, benzimidazolyl, or benzothiazolyl.

In some cases, q is 0. In some of these cases, Z is C₀₋₁₀alkylene-COOR¹ (e.g., —COOR¹, —CH₂COOR¹, or —CH₂CH₂COOR¹), C₀₋₁₀alkylene-CN, C₀₋₁₀alkylene-NR¹₃⁺ (e.g., —NR¹₃⁺, —CH₂NR¹₃⁺, or —CH₂CH₂NR¹₃⁺), C₀₋₁₀alkylene-ONO₂ (e.g., —ONO₂, —CH₂ONO₂, —CH₂CH₂ONO₂), C₀₋₁₀alkylene-OSO₂R² (e.g., —OSO₂R², —CH₂OSO₂R², or —CH₂CH₂OSO₂R²), or C₀₋₁₀alkylene-OPO₃H₂ (e.g., —OPO₃H₂, —CH₂OPO₃H₂, —CH₂CH₂OPO₃H₂). In some of these embodiments, R¹ is H, methyl, ethyl, n-propyl, isopropyl, n-butyl, s-butyl, t-butyl, pentyl, or hexyl, and R² is OH, OMe, or NMe₂. In various embodiments, Z is C₁₋₁₀alkyl (e.g., methyl, ethyl, n-propyl, isopropyl, n-butyl, s-butyl, t-butyl, pentyl, or hexyl, heptyl, octyl, nonyl, or decyl), C₀₋₁₀alkylene-C₃₋₈cycloalkyl (e.g., -cyclopentyl, —CH₂cyclopentyl, —CH₂CH₂cyclopentyl, -cyclohexyl, —CH₂cyclohexyl, or —CH₂CH₂cyclohexyl), C₀₋₁₀alkylene-C₃₋₈heterocycloalkyl (e.g., -pyrrolidinyl, —CH₂pyrrolidinyl, —CH₂CH₂pyrrolidinyl, -pyrrolidonyl, —CH₂pyrrolidonyl, or —CH₂CH₂pyrrolidonyl), C₀₋₁₀alkylene-aryl (e.g., -Ph, —CH₂Ph, —CH₂CH₂Ph), or C₀₋₁₀alkylene-heteroaryl (e.g., -imdazolyl, —CH₂imdazolyl, —CH₂CH₂imdazolyl, -pyrrolyl, —CH₂pyrrolyl, —CH₂CH₂pyrrolyl, -thiophenyl, —CH₂thiophenyl, or —CH₂CH₂thiophenyl). In some embodiments, Z is C₂₋₁₀polyalkyleneoxide, C₂₋₁₀polyalkyleneoxide-C₃₋₈cycloalkyl, C₂₋₁₀polyalkyleneoxide-C₃₋₈heterocycloalkyl, C₂₋₁₀polyalkyleneoxide-aryl, C₂₋₁₀polyalkyleneoxide-heteroaryl, C₂₋₁₀polyalkyleneoxide-COOR¹, C₂₋₁₀polyalkyleneoxide-CN, C₂₋₁₀polyalkyleneoxide-NR¹₂, C₂₋₁₀polyalkyleneoxide-NR¹₃⁺, C₂₋₁₀polyalkyleneoxide-ONO₂, C₂₋₁₀polyalkyleneoxide-OSO₂R², or C₂₋₁₀polyalkyleneoxide-OPO₃H₂. In various embodiments, Z is

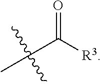

In some of these embodiments, R³ is C₁₋₁₀alkyl (e.g., methyl, ethyl, n-propyl, isopropyl, n-butyl, s-butyl, t-butyl, pentyl, or hexyl, heptyl, octyl, nonyl, or decyl), C₀₋₁₀alkylene-C₃₋₈cycloalkyl (e.g., -cyclopentyl, —CH₂cyclopentyl, —CH₂CH₂cyclopentyl, -cyclohexyl, —CH₂cyclohexyl, or —CH₂CH₂cyclohexyl), C₀₋₁₀alkylene-C₃₋₈heterocycloalkyl (e.g., -pyrrolidinyl, —CH₂pyrrolidinyl, —CH₂CH₂pyrrolidinyl, -pyrrolidonyl, —CH₂pyrrolidonyl, or —CH₂CH₂pyrrolidonyl), C₀₋₁₀alkylene-aryl (e.g., -Ph, —CH₂Ph, or —CH₂CH₂Ph), C₀₋₁₀alkylene-heteroaryl (e.g., -imdazolyl, —CH₂imdazolyl, —CH₂CH₂imdazolyl, -pyrrolyl, —CH₂pyrrolyl, —CH₂CH₂pyrrolyl, -thiophenyl, —CH₂thiophenyl, or —CH₂CH₂thiophenyl), C₂₋₁₀polyalkyleneoxide, C₂₋₁₀polyalkyleneoxide-C₃₋₈cycloalkyl, C₂₋₁₀polyalkyleneoxide-C₅₋₈heterocycloalkyl, C₂₋₁₀polyalkyleneoxide-aryl, or C₂₋₁₀polyalkyleneoxide-heteroaryl. In some embodiments, Z can be

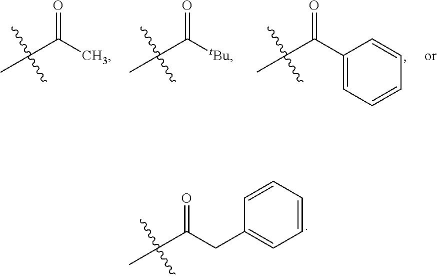

In some embodiments, R³ is C₁₋₁₀alkylene-COOR⁴, C₀₋₁₀alkylene-OR⁴, C₀₋₁₀alkylene-SR⁴, C₀₋₁₀alkylene-NR¹₂, C₀₋₁₀alkylene-NR¹₃⁺, C₁₋₁₀alkylene-CN, C₁₋₁₀alkylene-NO₂, C₁₋₁₀alkylene-ONO₂, C₁₋₁₀alkylene-SO₂R², C₁₋₁₀alkylene-OSO₂R², C₀₋₁₀alkylene-PO₃H₂, or C₀₋₁₀alkylene-OPO₃H₂. In some cases, R³ is C₂₋₁₀polyalkyleneoxide, C₂₋₁₀polyalkyleneoxide-C₃₋₈cycloalkyl, C₂₋₁₀polyalkyleneoxide-C₅₋₈heterocycloalkyl, C₂₋₁₀polyalkyleneoxide-aryl, C₂₋₁₀polyalkyleneoxide-heteroaryl, C₂₋₁₀polyalkyleneoxide-COOR⁴, C₂₋₁₀polyalkyleneoxide-OR⁴, C₂₋₁₀polyalkyleneoxide-SR⁴, C₂₋₁₀polyalkyleneoxide-NR¹₂, C₂₋₁₀polyalkyleneoxide-NR¹₃⁺, C₂₋₁₀polyalkyleneoxide-CN, C₂₋₁₀polyalkyleneoxide—NO₂, C₂₋₁₀polyalkyleneoxide-SO₃R⁴, or C₂₋₁₀polyalkyleneoxide-PO₃H₂.

In various cases, q is 1. In some of these cases, X is C₁₋₁₀alkylene (e.g., methylene, ethylene, propylene, butylene, pentylene, hexylene, heptylene, octylene, nonylene, or decylene), such as C₁₋₆alkylene or C₁₋₄alkylene. For example X can be —CH₂— or —CH₂CH₂—. In some cases, X is arylene (e.g., phenylene, naphthylene, tetrahydronaphthylene, phenanthrenylene, biphenylenylene, indanylene, indenylene, anthracenylene, and fluorenylene). For example, X can be phenylene, naphthylene, or anthracenylene. In various embodiments, X is C₂₋₁₀polyalkyleneoxide, such as C₂₋₆polyalkyleneoxide or C₂₋₄polyalkyleneoxide. In some cases, the polyalkyleneoxide is polyethyleneoxide, polypropyleneoxide, or polybutyleneoxide. In various cases, X is absent.

In some embodiments, Y is O. In various embodiments, Y is S. In some cases, Y is NR¹. In some embodiments, R¹ is H. In some cases, R¹ is C₁₋₆alkyl (e.g., methyl, ethyl, propyl, butyl, pentyl, or hexyl). For example, Y can be NMe, NEt, or NPr.

In various cases, Z is H. In some of these embodiments, X—Y—Z is —CH₂OH, —CH₂NH₂, or —CH₂SH. In some embodiments, Z is C₁₋₁₀alkylene-ONO₂ (e.g., CH₂ONO₂ or CH₂CH₂ONO₂), C₁₋₁₀alkylene-OSO₂R² (e.g., CH₂OSO₂R² or CH₂CH₂OSO₂R²) or C₁₋₁₀alkylene-OPO₃H₂ (e.g., CH₂OPO₃H₂ or CH₂CH₂OPO₃H₂). In some of these embodiments, R² is OR¹, and R¹ is H, methyl, ethyl, n-propyl, isopropyl, n-butyl, s-butyl, or t-butyl (e.g., OH, OMe, OEt, OPr, OⁱPr, OⁿBu, O^{s}Bu, or O^{t}Bu). For example, Z can be —CH₂OSO₃H, —CH₂OSO₃Me, —CH₂OSO₃Et, and —CH₂OSO₃^{t}Bu. In some cases, R² is NR¹₂, and each R¹ independently is H, methyl, or phenyl (e.g., NH₂, NHMe, NMe₂, NHPh, and NMePh). For example, Z can be —CH₂OSO₂NH₂, —CH₂OSO₂NHMe, —CH₂OSO₂NMe₂, —CH₂OSO₂NHPh, and —CH₂OSO₂NMePh. In some cases, Z is C₁₋₁₀alkyl (e.g., methyl, ethyl, n-propyl, isopropyl, n-butyl, s-butyl, or t-butyl, pentyl, hexyl, heptyl, octyl, nonyl, or decyl), C₀₋₁₀alkylene-C₃₋₈cycloalkyl (e.g., -cyclopentyl, —CH₂cyclopentyl, —CH₂CH₂cyclopentyl, -cyclohexyl, —CH₂cyclohexyl, or —CH₂CH₂cyclohexyl), C₀₋₁₀alkylene-C₅₋₈heterocycloalkyl (e.g., -pyrrolidinyl, —CH₂pyrrolidinyl, —CH₂CH₂pyrrolidinyl, -pyrrolidonyl, —CH₂pyrrolidonyl, or —CH₂CH₂pyrrolidonyl), C₀₋₁₀alkylene-aryl (e.g., -Ph, —CH₂Ph, or —CH₂CH₂Ph), or C₀₋₁₀alkylene-heteroaryl (e.g., -imdazolyl, —CH₂imdazolyl, —CH₂CH₂imdazolyl, -pyrrolyl, —CH₂pyrrolyl, —CH₂CH₂pyrrolyl, -thiophenyl, —CH₂thiophenyl, or —CH₂CH₂thiophenyl). In various embodiments, Z is C₅H₁₁, —CH₂Ph,

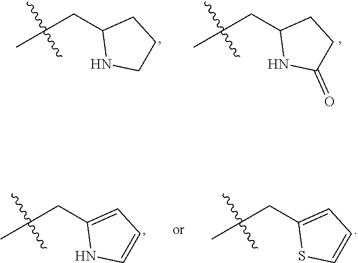 [SMILES: *CC1CCCN1 ; *CC1CCC(=O)N1 ; *Cc1ccc[nH]1 ; *Cc1cccs1]

In some cases, Z is C₀₋₁₀alkylene-COOR¹, C₀₋₁₀alkylene-CN, C₁₋₁₀alkylene-OR¹, C₁₋₁₀alkylene-SR¹, C₁₋₁₀alkylene-NR¹₂, or C₁₋₁₀alkylene-NR¹₃⁺, and R¹ can be H, methyl, ethyl, n-propyl, isopropyl, n-butyl, s-butyl, or t-butyl. For example, Z can be —CH₂OH, —CH₂NH₂, —CH₂N(CH₃)3⁺, or —CH₂COOH. In some cases Z is C₂₋₁₀polyalkyleneoxide, C₂₋₁₀polyalkyleneoxide-C₃₋₈cycloalkyl, C₂₋₁₀polyalkyleneoxide-C₃₋₈heterocycloalkyl, C₂₋₁₀polyalkyleneoxide-aryl, C₂₋₁₀polyalkyleneoxide-heteroaryl, C₂₋₁₀polyalkyleneoxide-COOR¹, C₂₋₁₀polyalkyleneoxide-CN, C₂₋₁₀polyalkyleneoxide-OR¹, C₂₋₁₀polyalkyleneoxide-SR¹, C₂₋₁₀polyalkyleneoxide-NR¹₂, C₂₋₁₀polyalkyleneoxide-NR¹₃⁺, C₂₋₁₀polyalkyleneoxide-ONO₂, C₂₋₁₀polyalkyleneoxide-OSO₂R², or C₂₋₁₀polyalkyleneoxide-OPO₃H₂. In some embodiments, Z is

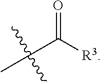

In some of these embodiments, R³ is C₁₋₁₀alkyl, C₀₋₁₀alkylene-C₃₋₈cycloalkyl (e.g., -cyclopentyl, —CH₂cyclopentyl, —CH₂CH₂cyclopentyl, -cyclohexyl, —CH₂cyclohexyl, or —CH₂CH₂cyclohexyl), C₀₋₁₀alkylene-C₅₋₈heterocycloalkyl (e.g., -pyrrolidinyl, —CH₂pyrrolidinyl, —CH₂CH₂pyrrolidinyl, -pyrrolidonyl, —CH₂pyrrolidonyl, —CH₂CH₂pyrrolidonyl)), C₀₋₁₀alkylene-aryl (e.g., -Ph, —CH₂Ph, or —CH₂CH₂Ph), C₀₋₁₀alkylene-heteroaryl (e.g., -imdazolyl, —CH₂imdazolyl, —CH₂CH₂imdazolyl, -pyrrolyl, —CH₂pyrrolyl, —CH₂CH₂pyrrolyl, -thiophenyl, —CH₂thiophenyl, —CH₂CH₂thiophenyl), C₂₋₁₀polyalkyleneoxide, C₂₋₁₀polyalkyleneoxide-C₃₋₈cycloalkyl, C₂₋₁₀polyalkyleneoxide-C₅₋₈heterocycloalkyl, C₂₋₁₀polyalkyleneoxide-aryl, or C₂₋₁₀polyalkyleneoxide-heteroaryl). For example, Z can be

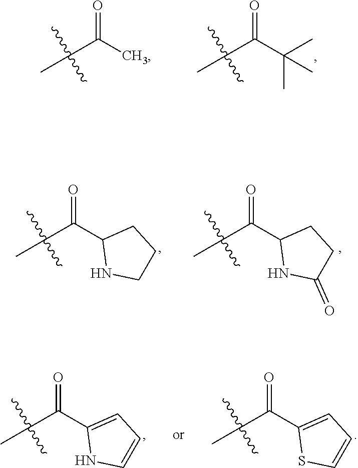

In some embodiments, R³ is C₁₋₁₀alkylene-COOR⁴, C₀₋₁₀alkylene-OR⁴, C₀₋₁₀alkylene-SR⁴, C₀₋₁₀alkylene-NR¹₂, C₀₋₁₀alkylene-NR¹₃⁺ (e.g., —CH₂N(CH₃)3+or —CH₂CH₂N(CH₃)₃⁺), C₁₋₁₀alkylene-CN, C₁₋₁₀alkylene-NO₂, C₁₋₁₀alkylene-ONO₂, C₁₋₁₀alkylene-SO₂R², C₁₋₁₀alkylene-OSO₂R², C₁₋₁₀alkylene-PO₃H₂, or C₁₋₁₀alkylene-OPO₃H₂. In some cases, R³ is CH(NR¹₂)R⁵. In some of these embodiments, R⁵ is H, C₁₋₆alkyl, C₁₋₆alkylene-aryl, or C₁₋₆alkylene-heteroaryl. For example, R⁵ can include —H, —CH₃, —CH(CH₃)₂, —CH₂CH(CH₃)₂, —CH(CH₃)(CH₂CH₃), —CH₂Ph, —CH₂Ph(OH). In various embodiments, R⁵ is C₁₋₆alkylene-OR¹, C₁₋₆alkylene-SR¹, C₁₋₆alkylene-COOH, C₁₋₆alkylene-CONR¹₂, C₁₋₆alkylene-NR¹₂, or C₁₋₆alkylene-NHC(NH₂)₂. For example, R⁵ is —CH₂OH, —CH(CH₃)OH, —CH₂SH, —CH₂CH₂SCH₃, —CH₂COOH, —CH₂CH₂COOH, —CH₂CONH₂, —CH₂CH₂CONH₂, —(CH₂)₄NH₂, or —(CH₂)₃NHC(NH₂)NH₂⁺.

In some embodiments, the polymer comprises structural units selected from the group consisting of:

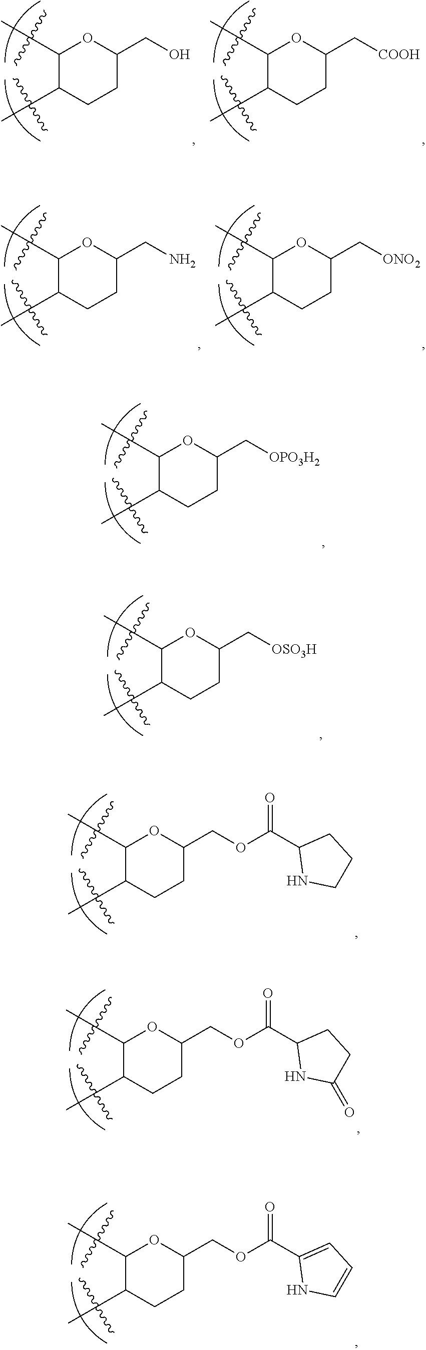

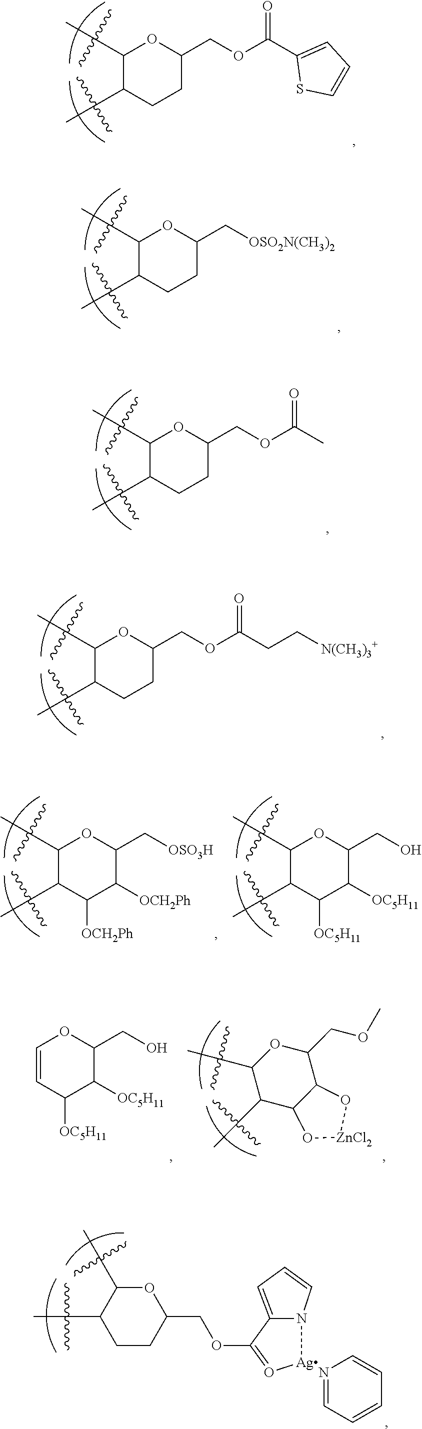

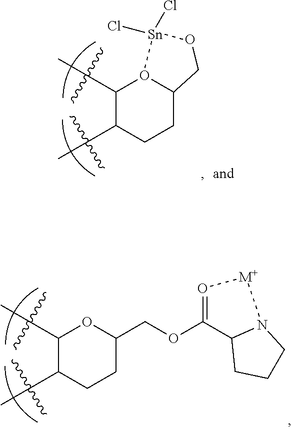

or a combination thereof, wherein M is Zn²⁺, Sn²⁺, Sn⁴⁺, or Ag⁺.

In some cases, p+r is at least 10, or at least 15, or at least 20, or at least 25, or at least 30, or at least 35, or at least 40, or at least 45, or at least 50, or at least 55, or at least 60, or at least 65, or at least 70, or at least 75, or at least 80, or at least 85, or at least 90, or at least 95, or at least 100, or at least 110, or at least 115, or at least 120, or at least 125, or at least 130, or at least 135, or at least 140, or at least 145, or at least 150, or at least 155, or at least 160, or at least 165, or at least 170, or at least 175, or at least 180, or at least 185, or at least 190, or at least 195, or at least 200. In various cases, p+r is 400 or less, or 390 or less, or 380 or less, or 370 or less, or 360 or less, or 350 or less, or 340 or less, or 330 or less, or 320 or less, or 310 or less, or 300 or less, or 290 or less, or 280 or less, or 270 or less, or 260 or less, or 250 or less, or 240 or less, or 230 or less, or 220 or less, or 210 or less, or 200 or less, or 190 or less, or 180 or less, or 170 or less, or 160 or less, or 150 or less, 140 or less, or 130 or less, or 120 or less, or 110 or less, or 100 or less. For example, p+r can be in a range of 5 to 400, or 10 to 300, or 25 to 200, or 50 to 100, such as from 10 to 100 or 30 to 150.

In some embodiments, the cyclic ether polymers further include one or more structural units of Formula (II′):

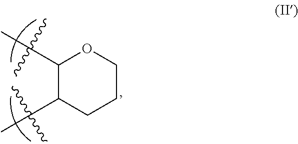

wherein the polymer comprises no more than 70% of structural units of Formula (II′). In some cases, Formula (II′) comprises no more than 65%, or no more than 60%, or no more than 55%, or no more than 50%, or no more than 45%, or no more than 40%, or no more than 35%, or no more than 30%, or no more than 25%, or no more than 20%, or no more than 15%, or no more than 10%, or no more than 5%, or no more than 1% of structual units of Formula (II′). For example, Formula (II′) could comprise between 1% and 70%, or between 5% and 50%, or between 10% and 25% of the total structural units in the polymer.

In various cases, the cyclic ether polymers are copolymers. In these embodiments, the copolymer can include a polyolefin. The polyolefin be derived from a monomer selected from the group consisting of acrylamide, acrylate, acrylic acid and derivatives or salts thereof, acrylohalide, acrylonitrile, allyl alcohol, allyl ether, allyl ester, allyl carbonate, allyl carbamate, allyl sulfone, allyl sulfonic acid, allyl amine, allyl cyanide, vinyl ester, vinyl thioester, vinyl pyrrolidone, α-olefin, styrene, and combinations thereof. In some embodiments, the copolymer can be derived from a monomer selected from the group consisting of ethylene, styrene, (C₀₋₂alkyl)acrylamide (e.g., methacrylamide, ethylacrylamide, N,N-dimethylacrylamide, or N-isopropylacrylamide), (C₀₋₂alkyl)acrylate (e.g., methyl acrylate, ethyl acrylate, ethyl methacrylate, or 2-hydroxyethyl methacrylate), and (C₀₋₂alkyl)acrylic acid or a derivative thereof (e.g., acrylic acid or methacrylic acid). In some embodiments, the cyclic ether polymer further includes methacrylate.

The polymers disclosed herein include at least 11 optionally substituted carbon-linked tetrahydropyran rings. In one embodiment the polymer comprises at least 15 optionally substituted carbon-linked tetrahydropyran rings. In one embodiment the polymer comprises at least 20 optionally substituted carbon-linked tetrahydropyran rings. In one embodiment the polymer comprises at least 30 optionally substituted carbon-linked tetrahydropyran rings. In one embodiment the polymer comprises at least 50 optionally substituted carbon-linked tetrahydropyran rings. In one embodiment the polymer comprises at least 100 optionally substituted carbon-linked tetrahydropyran rings. In one embodiment the polymer comprises at least 200 optionally substituted carbon-linked tetrahydropyran rings. In one embodiment the polymer comprises at least 300 optionally substituted carbon-linked tetrahydropyran rings. In one embodiment the polymer comprises at least 400 optionally substituted carbon-linked tetrahydropyran rings. In one embodiment the polymer comprises up to about 500 optionally substituted carbon-linked tetrahydropyran rings. In one embodiment the polymer comprises up to about 400 optionally substituted carbon-linked tetrahydropyran rings. In one embodiment the polymer comprises up to about 300 optionally substituted carbon-linked tetrahydropyran rings. In one embodiment the polymer comprises up to about 200 optionally substituted carbon-linked tetrahydropyran rings. In one embodiment the polymer comprises up to about100 optionally substituted carbon-linked tetrahydropyran rings. In one embodiment the polymer comprises up to about 50 optionally substituted carbon-linked tetrahydropyran rings. In one embodiment the polymer comprises up to about 30 optionally substituted carbon-linked tetrahydropyran rings. In one embodiment the polymer comprises up to about 20 optionally substituted carbon-linked tetrahydropyran rings. In one embodiment the polymer comprises up to about 15 optionally substituted carbon-linked tetrahydropyran rings. The polymers described herein can also be oligomers, which include about 2, 3, 4, 5, 6, 7, 8, 9, or 10 optionally substituted carbon-linked tetrahydropyran rings. In one embodiment the oligomer comprises up to about 5 optionally substituted carbon-linked tetrahydropyran rings. In one embodiment the oligomer comprises up to about 10 optionally substituted carbon-linked tetrahydropyran rings. In one embodiment the oligomer comprises less than about 10 optionally substituted carbon-linked tetrahydropyran rings. In one embodiment the oligomer comprises less than about 8 optionally substituted carbon-linked tetrahydropyran rings. In one embodiment the oligomer comprises less than about 6 optionally substituted carbon-linked tetrahydropyran rings.

### Synthesis of Cyclic Ether Monomers and Polymers

The substituted cyclic ether monomers described herein are can be synthesized by any method known to one skilled in the art. See, e.g., Abbas and Abdelaal, Polymer Bulletin, 36:273-278 (1996), Abdelaal, Polymer Chemistry, 40:3909-3915 (2002), and Vidal, Bioorganic and Medicinal Chemistry 10:4051 (2002). For example, 2-hydroxymethyl-3,4-dihydro-2H-pyran can be derivatized at the 2-hydroxymethyl substituent through reactions well known to those skilled in the art, such as, for example, oxidation, esterification, substitution, and sulfonation, to form the monomers described herein, a shown in Scheme 1, below.

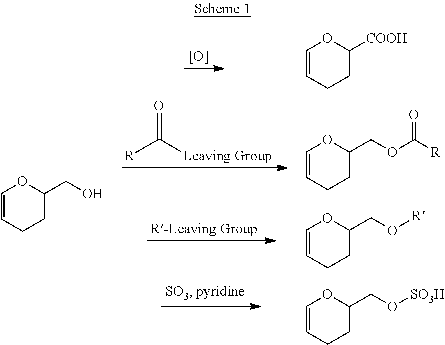

The substituted cyclic ether polymers described herein can be synthesized by any method known to one skilled in the art. See, e.g., Winston, M. S., et al. Angewandte Communications, 51(39):9822-9824 (2012). For example, a substituted cyclic ether monomer of Formula (I) can be reacted with a Lewis acid catalyst to form a polymer of Formula (II) in high yield, as shown in Scheme 2, below. The resulting polymer can be characterized by, for example, NMR spectroscopy, infrared spectroscopy, and low resolution mass spectrometry (LRMS). This synthetic method allows access to polymers having varying polarity and affinity for non-keratinized surfaces of the oral cavity and/or the oropharynx, such as surfaces that are lined with carbohydrate-based molecules (e.g., glycoproteins).

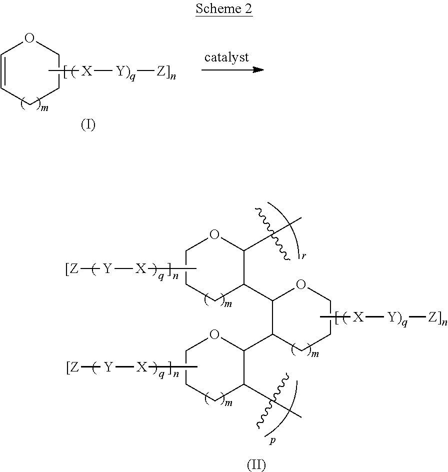

In some embodiments, the catalyst can include a d-block transition metal. Suitable d-block transition metals include palladium, platinum, and iron. In some cases, the catalyst can include a p-block metal. Suitable p-block metals include boron and aluminum. In various cases, the catalyst further includes one or more coordinated solvent molecules. Suitable solvents include acetonitrile, tetrahydrofuran, trifluoroethanol, methylene chloride, and chloroform. In various embodiments, the catalyst is selected from the group consisting of BF₃.OEt₂, FeBr₃, [(DAB)Pd(CH₃CN)_{2]}²⁺ (DAB=2,3-bis(2,6-diisopropylphenylimino)butane), and toluene sulfonic acid. The catalyst can be present in any amount sufficient to elicit polymerization. In some embodiments, the catalyst is present in an amount in a range of 1 to 10 mol %, or 2 to 8 mol % or 3 to 6 mol %, or 1 to 5 mol %, or 2 to 10 mol %.

Nonlimiting examples of the polymers disclosed herein include:

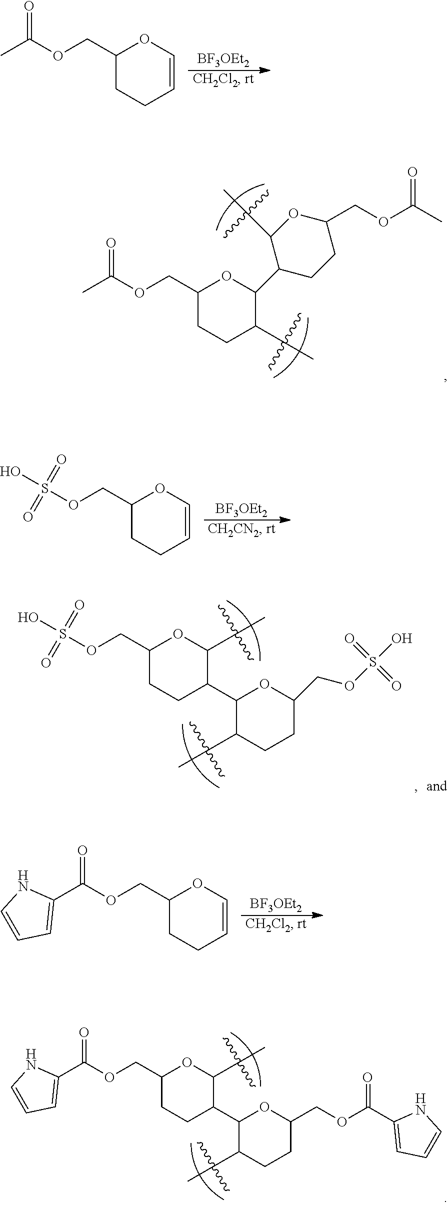

Additional synthetic procedures for preparing the monomers and polymers disclosed herein can be found in the Examples section.

### Drug Conjugates

Yet another aspect of the disclosure provides drug conjugates of the monomers of Formula (I), the polymers having structural units of Formula (II), and pharmaceutically acceptable salts and solvates thereof. The drug conjugates disclosed herein are capable of cleaving in the oral cavity and/or oropharynx upon contact with water, enzymes (e.g., esterases or salivary enzymes), and other reactive agents in the oral cavity, such as fluoride. Therefore, the drug conjugates disclosed herein can be used to deliver therapeutic agents to the oral cavity and/or oropharynx.

In this aspect, a therapeutic agent and a monomer or polymer, as described herein, are attached through a linking group selected from the group consisting of an ester linkage, a thioester linkage, an amide linkage, a carbamate linkage, a carbonate linkage, and a metal ligand bond.

In some cases, the linking group is formed from a reactive substituent on the Z group of the monomer or polymer and a reactive group on the therapeutic agent. In some embodiments, the reactive substituent on the Z group includes a hydroxyl, a thiol, an amino, a carboxylic acid, or an activated carboxylic acid. An activated carboxylic acid is a carboxylic acid that has been derivatized to include a leaving group, such as an acyl chloride, anhydride, or ester (e.g., include N-hydroxysuccinimide (NHS), tosylate (Tos), mesylate, and triflate).

The therapeutic agent can be any small molecule, peptide, or protein (including an antibody) that has a reactive group capable of reacting with Z of the monomers of Formula (I) or the polymers comprising structural units of Formula (II) to result in an ester linkage, a thioester linkage, an amide linkage, a carbamate linkage, a carbonate linkage, or a metal ligand bond. When the therapeutic agent includes a hydroxyl group, a thiol group, and/or an amino group, it can react with a Z group on Formula (I) or (II) that includes a carboxylic acid or activated carboxylic acid. When the therapeutic agent includes a carboxylic acid or activated carboxylic acid, it can react with a Z group on Formula (I) or (II) that includes a hydroxyl group, a thiol group, and/or an amino group. When each of the Z group of Formula (I) or (II) and the therapeutic agent include a metal-binding ligand (e.g., nitrogen, oxygen, sulfur, carboxylate), then the drug conjugate can be formed by complexing the compound of Formula (I) or (II) and the therapeutic agent to the same metal. Suitable metals for complexation include an f-block metal (e.g., cerium or lanthanum), a d-block metal (e.g., zinc or silver), a p-block metal (e.g., tin), or an alkaline earth metal (e.g., magnesium or calcium) as previously described herein.

In some embodiments, the therapeutic agent is an analgesic, anesthetic, antiobiotic, antifungal agent, anticancer agent, antiviral agent, anti-inflammatory agent, or steroid. The analgesic can be selected from the group consisting of acetylsalicylic acid, choline magnesium salicylate, diflunisal, magnesium salicylate, salsalate, sodium salicylate, diclofenac, etodolac, fenoprofen, flurbiprofen, indomethacin, ketoprofen, ketorolac, meclofenamate, naproxen, nabumetone, phenylbutazone, piroxicam, sulindac, tolmetin, acetaminophen, ibuprofen, a Cox-2 inhibitor, and tramadol. For example, the analgesic can be ibuprofen. The anesthetic can be selected from the group consisting of lidocaine, benzocaine, and tetracaine. The antibiotic can be selected from the group consisting of penicillin, metronidazole, amoxicillin, ampicillin, tetracycline, and doxycycline. The antifungal agent can be selected from the group consisting of fluconazole, ketoconazole, miconazole, and itraconazole. The anticancer agent is selected from the group consisting of doxorubicin, cisplatin, 5-fluorouracil, carboplatin, bleomycin, methotrexate, docetaxel, paclitaxel, and gemcitabine. The steroid can be selected from the group consisting of hydrocortisone, hydrocortisone acetate, cortisone, cortisone acetate, tixocortol pivalate, prednisolone, methylprednisolone, and prednisone, triamcinolone acetonide, triamcinolone alcohol, mometasone, amcinonide, budesonide, desonide, fluocinonide, fluocinolone acetonide, halcinonide, betamethasone and derivatives thereof, betamethasone sodium phosphate, dexamethasone, dexamethasone sodium phosphate, fluocortolone, hydrocortisone-17-valerate, halometasone, alclometasone dipropionate, betamethasone valerate, betamethasone dipropionate, prednicarbate, clobetasone-17-butyrate, clobetasol-17-propionate, fluocortolone caproate, fluocortolone pivalate, fluprednidene acetate, hydrocortisone-17-valerate, halometasone, alclometasone dipropionate, betamethasone valerate, betamethasone dipropionate, prednicarbate, clobetasone-17-butyrate, clobetasol-17-propionate, fluocortolone caproate, fluocortolone pivalate, and fluprednidene acetate.

In some embodiments, the drug conjugate is selected from the group consisting of:

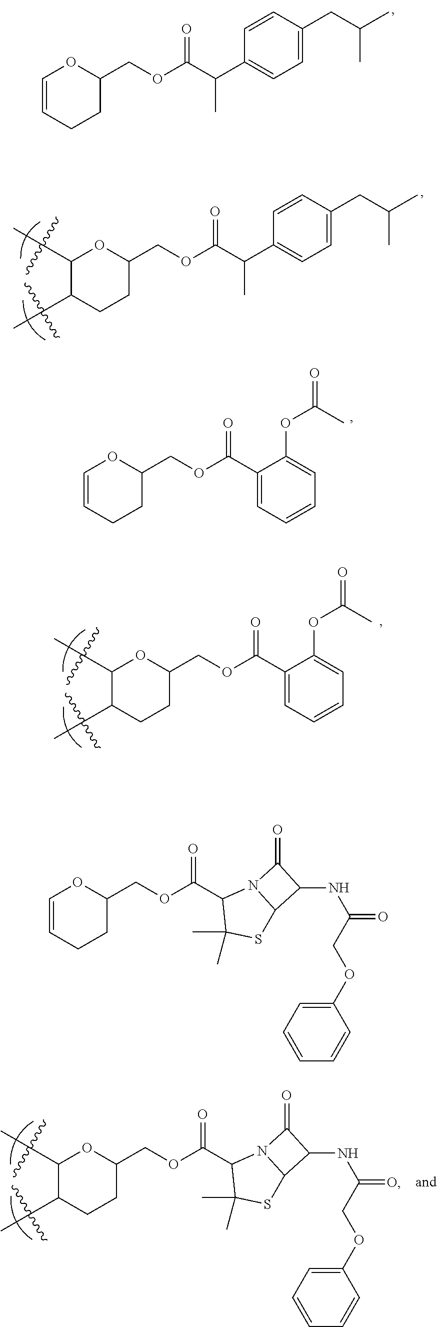

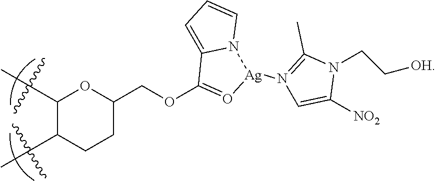

The drug conjugates described herein can be formed by any method known in the art for synthesizing ester, thioester, amide, carbamate, or carbonate linkages, or for complexing ligands to metals. See, e.g., Greg T. Hermanson, Bioconjugate Techniques, Academic Press (1996)).

In some embodiments, the drug conjugate is formed via a nucleophilic substitution reaction between a nucleophile (e.g., a hydroxyl group, an amino group, or a thiol group) on Z and an electrophile having a leaving group on the therapeutic agent (e.g., carboxylic acid or activated carboxylic acid). In various embodiments, the drug conjugate is formed via a nucleophilic substitution reaction between a nucleophile (e.g., a hydroxyl group, an amino group, or a thiol group) on the therapeutic agent and an electrophile having a leaving group on Z (e.g., carboxylic acid or activated carboxylic acid). In some cases, the drug conjugate can be formed by coordinating a metal-binding ligand on each of Z and the therapeutic agent to a metal.

For example, a drug conjugate that includes ibuprofen can be formed, as shown below:

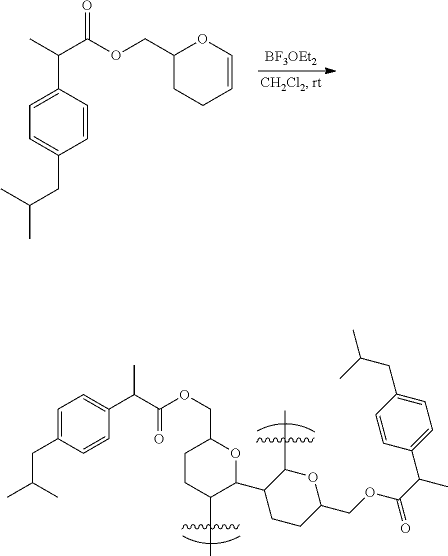

### Mucoadhesive Formulations

Still another aspect of the disclosure provides mucoadhesive formulations that include a monomer of Formula (I), a polymer of Formula (II), and/or a drug conjugate thereof, as previously described herein. The mucoadhesive formulations described herein can adhere to non-keratinized surfaces of the oral cavity and/or the oropharynx, such as surfaces that are lined with carbohydrate-based molecules (e.g., glycoproteins). Surfaces to which the mucoadhesive formulations can adhere included, for example, the lips, gingivae, retromolar trigone, teeth, hard palate, cheek mucosa, mobile tongue, floor of the mouth, the palatine tonsils, soft palate, tongue base, and posterior pharyngeal walls. When applied to oral surfaces, the mucoadhesive formulations can have a local barrier effect (such as an impermeable backing of a patch), providing protection, inhibiting irritation, and/or accelerating healing of tissue, such as inflamed or damaged tissue. As such, the formulations disclosed herein can be used to treat conditions of the oral cavity and/or oropharynx, such as mucositis, gum (periodontal) disease, necrotizing ulcerative gingivitis, necrotizing ulcerative periodontitis, and cellulitis. Further, the formulations also can be used as guided tissue regeneration (GTR) films, which are used in oral surgery to regenerate tissues in the oral cavity and/or oropharynx.

When the mucoadhesive formulations include a drug conjugate, they can act as drug delivery agents to the oral cavity and/or oropharynx. Therefore, the formulations disclosed herein also are useful for treating diseases of the oral cavity and/or oropharynx, such as infections (e.g., bacterial, fungal, viral) and head and neck cancer. For example, when a mucoadhesive formulation containing a drug is applied to a surface in the oral cavity, and/or oropharynx, the therapeutic agent can be released from the conjugate through contact with water, enzymes (e.g., esterases or salivary enzymes), and other reactive agents in the oral cavity, such as fluoride. In some embodiments, the therapeutic agent can be released from the conjugate at a controlled rate for improved therapeutic outcome. The mucoadhesive drug delivery formulations disclosed herein are advantageous over traditional mucoadhesive drug delivery formulations because the therapeutic agents are covalently bound to a polymer forming the mucoadhesive formulation and then released via chemical reaction (e.g., ester hydrolysis). In contrast, traditional mucoadhesive films (e.g., GELCLAIR and MUGUARD) and hydrogels (e.g., PERIOCHIP and ARRESTIN) are typically not bio-active themselves, but contain active agents dispersed within that are slowly released from the film through diffusion. See also Morales et al., European Journal of Pharmaceutical Sciences and Biopharmaceutics 77:187-199 (2011).

The monomers, polymers, and drug conjugates described herein are particularly suited for mucoadhesive applications because they include numerous hydrophilic groups, such as hydroxyl, carboxyl, amide, amino, sulfate, and phosphate groups, which can attach to mucous or the cell membrane by various interactions, such as hydrogen bonding, hydrophobic interactions, and/or electrostatic interactions. Also, the hydrophobic core structure of the polymer will provide an amphiphilic effect with these hydrophilic pendant groups. The amphiphilic polymers can also engage in van der Waals forces to increase adhesion to mucosal surfaces. Furthermore, the charge, polarity, molecular weight, and length of the polymers and drug conjugates provided herein can be tailored to bond to oral surfaces having differing physiological properties, such as diseased tissue. See, e.g., Shaikh et al., J. Pharm. Bioallied Sci. 31(1):89-100 (2011).

The mucoadhesive formulations described herein can be in any form capable of adhering to a surface of the oral cavity and/or oropharynx. Suitable mucoadhesive forms include films, ointments, pastes, gels, varnishes, patches, and sprays. Methods of preparing these dosage forms are well known in the art. See, e.g., Remington: The Science and Practice of Pharmacy, 19th edition, (ed. A R Gennaro), Mack Publishing Co., Easton, Pa., 1995. In some cases, the formulation is a film which can adhere to surfaces in the oral cavity and/or oropharynx for prolonged periods of time (e.g. hours to days). In some cases, the films can be any thickness useful for coating or delivering a therapeutic agent to the oral cavity and/or oropharynx. For example, the films disclosed herein can have a thickness of about 50 and 400 microns, or about 100 and 300 microns, or about 200 to 300 microns. Methods of manufacturing and characterizing mucoadhesive films are well known to those skilled in the art, and can be found in Morales and McConville, European Journal of Pharmaceutics and Biopharmaceutics, 77:187-199 (2011).

The formulations described herein also can be in the form of ointments, pastes, and gels. The viscous nature of such dosage forms allows them to be retained on the surface of the oral cavity and/or oropharynx for minutes to hours.

The mucoadhesive formulations disclosed herein can further include a pharmaceutically acceptable excipient, preferably for topical administration, such as one or more of the following: permeation enhancer (e.g., sorption promoters or accelerants that penetrate into skin to reversibly decrease the barrier resistance, such as sulfoxides (DMSO), azones (laurocapram), pyrrolidones (2-pyrrolidone), alcohols and alkanols (ethanol or decanol), glycols (propylene glycol), surfactants, and terpenes), viscosity-increasing agent; surfactant; stabilizing agent/preservative; flavor, fragrance, sweetening agent; bioadhesive; an/or co-solubilizer. Examples of suitable excipients are described in Remington: The Science and Practice of Pharmacy, 19th edition, (ed. A R Gennaro), Mack Publishing Co., Easton, Pa., 1995.

The mucoadhesive formulations disclosed herein also can include one or more other active ingredients dispersed within the formulation, such as an antibacterial, disinfectant, antifungal, analgesic, anesthetic, antibiotic, antifungal agent, anticancer agent, antiviral agent, anti-inflammatory agent, steroid, and the like.

### Methods

The polymers and formulations disclosed herein can adhere to surfaces of the oral cavity and/or oropharynx to provide a protective layer over, e.g., oral mucosa. This protective layer can result in pain relief by soothing oral lesions of various etiologies, such as oral mucositis and/or stomatitis that can result from chemotherapy or radiation therapy, irritation due to oral surgery, periodontitis, gingivitis, necrotizing ulcerative gingivitis, necrotizing ulcerative periodontitis, periocoronitis, cellulitis, and traumatic ulcers caused by braces or ill-fitting dentures. When conjugated to a therapeutic agent, the polymers and formulations thereof can be used to deliver the therapeutic agent to the oral cavity and/or oropharynx, thereby enabling the treatment of oral diseases and conditions.

As such, provided herein is a method of treating a disease or condition in a subject comprising contacting a non-keratinized surface in the oral cavity and/or oropharynx of the subject with a polymer of Formula (II), a drug conjugate thereof, or a formulation thereof to provide a therapeutic benefit on the disease or condition to the subject. In some embodiments, the subject suffers from a disease or condition selected from the group consisting of head and neck cancer, mucositis, periodontitis, gingivitis, necrotizing ulcerative gingivitis, necrotizing ulcerative periodontitis, periocoronitis, candidosis, periodontal abscess, periapical abscess, cellulitis, benign oral cavity tumors, benign oropharyngeal tumors, leukoplakia, and erythroplakia. For example, the subject can suffer from mucositis. Mucositis, which is an inflammation of the mucous membranes in the mouth, is one of the most common oral problems occurring after chemotherapy and radiation therapy, and can contribute to oral infections, inability to taste normally and pain arising from the resulting open sores that can develop. In fact, mucositis can become so painful that the patient will not eat or drink, contributing to dehydration and malnutrition. The mucositis problem, however, is not restricted to cancer patients, as mucositis frequently also occurs in HIV patients, particularly when associated with Kaposi's sarcoma, in patients affected with non-Hodgkin's lymphoma, in debilitated elderly patients and in patients receiving BRM treatments like interleukin-2, TNF, interferons, lymphokine-activated lymphocytes and the like.

Further provided herein is a method for delivering a therapeutic agent to the oral cavity and/or oropharynx of a subject. This method includes contacting a non-keratinized surface of the oral cavity and/or oropharynx of the subject with the drug conjugate described herein or a formulation comprising the drug conjugate. A drug conjugate or formulation thereof can contact a non-keratinized surface of the oral cavity and/or oropharynx by administering the conjugate or formulation thereof to a subject, such as a human, in need thereof.

Use of the compounds and formulations disclosed herein, such as the substituted cyclic ether compounds of Formula (I), Formula (II), and drug conjugates thereof, to treat a disease or condition of the oral cavity and/or oropharynx, as well as use of the compounds in the preparation of a formulation for treating the disease or condition, also are contemplated.

In jurisdictions that forbid the patenting of methods that are practiced on the human body, the meaning of “administering” of a composition to a human subject shall be restricted to prescribing a controlled substance that a human subject will self-administer by any technique (e.g., orally, inhalation, topical application, injection, insertion, etc.). The broadest reasonable interpretation that is consistent with laws or regulations defining patentable subject matter is intended. In jurisdictions that do not forbid the patenting of methods that are practiced on the human body, the “administering” of compositions includes both methods practiced on the human body and also the foregoing activities.

### EXAMPLES

The following examples are provided for illustration and are not intended to limit the scope of the invention.

### Materials and Methods

The mucoadhesive viability of the polymers described herein can be determined by any method known to those skilled in the art, such as by the tensile method, the rotating disc method, the flow through method, and the rheological method, as described in, for example, Khutoryanskiy, Macromolecular Bioscience 11:748-764 (2011) and Morales and McConville, European Journal of Pharmaceutics and Biopharmaceutics, 77:187-199 (2011).

### Example 1

### Synthesis of Acetic Acid 3,4-dihydro-2H-pyran-2-ylmethyl ester Monomer

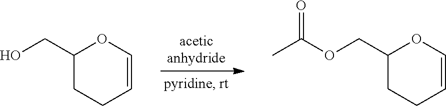

A small vial was charged with 3,4-dihydropyran-2H-pyran-2-methanol (200 mg, 1.75 mmol) and 0.330 mL (3.50 mmol) of acetic anhydride, and diluted with 1-2 mL of pyridine. The reaction was stirred at room temperature for 24 hours, and the solvent was diluted with toluene and removed under reduced pressure. The resulting oil was purified by silica gel chromatography (1:3 EtOAc/hexanes, R_{f} value of 0.6) to yield a clear liquid. 90% yield. ¹H NMR data (CDCl₃): 1H, 6.38 ppm; 1H, 4.78 ppm; 2H, 4.1 ppm; 1H 3.9 ppm; 3H, 2.1 ppm; 4H, 1.9-1.5 ppm.

### Example 2

### Synthesis of 2-(4-isobutyl-phenyl)-propionic acid 3,4-dihydro-2H-pyran-2-ylmethyl ester Monomer

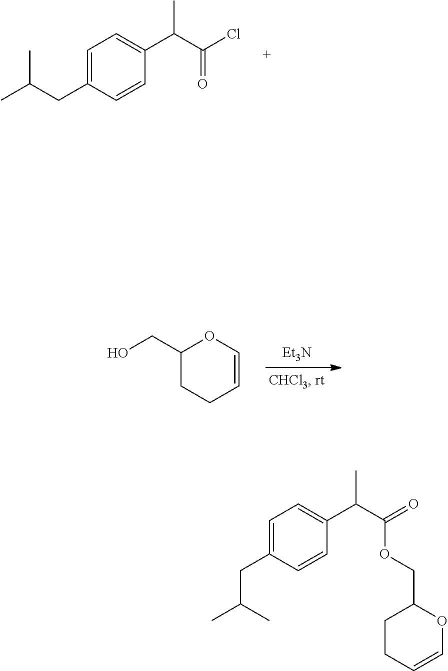 [SMILES: "Et3N" ; CC(C)Cc1ccc(C(C)C(=O)Cl)cc1 ; OCC1CCC=CO1 ; CC(C)Cc1ccc(C(C)C(=O)OCC2CCC=CO2)cc1]

A small vial was charged with 0.200 g (0.971 mmol) of ibuprofen and 2 mL of CH₃CN. To the clear solution was added 0.212 mL (2.91 mmol) of thionyl chloride, and the solution was allowed to stir for 20 hours at room temperature. The solvent was removed under reduced pressure, and to the resulting oil was added 0.971 mmol (0.110 g) of 3,4-dihydropyran-2H-pyran-2-methanol and 1 mL of triethylamine. The solution was diluted with 2 mL of CH₂Cl₂ and allowed to stir for 5 days at room temperature. The resulting solution was filtered, and the supernatant was removed under reduced pressure. The resulting dark brown oil was purified by column chromatography (silica gel, 1:3 EtOAC/hexanes) R_{f} value approximately 0.7. Yield 60-70%. ¹H NMR (CDCl₃): 2H, 7.20 ppm; 2H, 7.10 ppm; 1H, 6.38 ppm; 1H, 4.78 ppm; 2H, 4.2 ppm; 1H, 3.9 ppm; 1H, 3.7ppm; 2H 2.4 ppm; 3H, 1.41 ppm; 5H, 2.1-1.4; 6H, 0.95 ppm.

### Example 3

### Synthesis of 1H-Pyrrole-2-carboxylic acid 3,4-dihydro-2H-2-ylmethyl ester Monomer

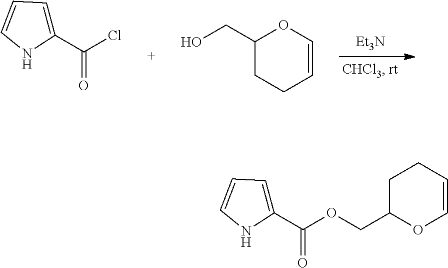 [SMILES: "Et3N" ; O=C(Cl)c1ccc[nH]1 ; OCC1CCC=CO1 ; O=C(OCC1CCC=CO1)c1ccc[nH]1]

To a small vial was added 2-pyrrole-2-carboxylic acid (0.200 g, 1.82 mmol) and 1.5 mL of SOCl₂, and the resulting solution was heated to 70° C. for 0.5 hrs. SOCl₂ was removed under reduced pressure to yield a reddish brown solid. The material was re-dissolved in 2 mL of toluene, and 0.248 g (2.18 mmol) of 3,4-dihydropyran-2H-pyran-2-methanol was added along with 1 mL of triethylamine. This mixture was heated to 60° C. and allowed to stir for 24 hours. A white precipitate developed, which was filtered, and the resulting solvent was removed from the supernatant to yield a brown oil. The brown oil was flashed through a silica gel column (1:3 EtOAC/hexanes) to yield the title compound (115 mgs, 35-40%) as a white solid. ¹H NMR data (CDCl₃): 1H, 9.20 ppm; 2H, 6.92 ppm; 1H, 6.38 ppm; 1H, 6.2 ppm; 1H, 4.75 ppm; 2H, 4.20ppm; 1H, 4.05 ppm; 4H, 2.2-1.75 ppm.

### Example 4

### Synthesis of dimethyl-sulfamic acid 3,4-dihydro-2H-pyran-2-ylmethyl ester Monomer

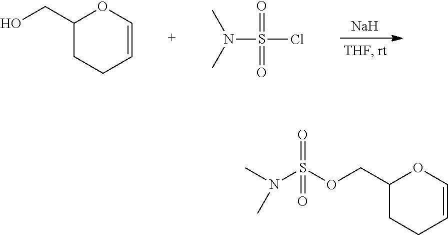

To a dried vial was added 3,4-dihydropyran-2H-pyran-2-methanol (0.050 g, 0.44 mmol), NaH (0.032 g, 1.33 mmol) and 4 mL of THF. To the resulting suspension was added N,N-dimethylsulfamoyl chloride (0.191 g, 1.33 mmol). The suspension was stirred for 1.5 days and a faint orange color developed. The solvent was removed under reduced pressure to yield a darker amber residue, which was washed with Et₂O. The resulting oil was soluble in MeOH. The product was carried forward without further purification for polymerization. Estimated yield 60-70%. ¹H NMR data (CDCl₃): 1H, 6.37 ppm; 1H, 4.72 ppm; 2H, 4.2 ppm; 1H, 4.10 ppm; 6H, 2.8 ppm; 4H, 2.20-1.75 ppm.

### Example 5

### Synthesis of poly(acetic acid 3,4-dihydro-2H-pyran-2-ylmethyl ester) Homopolymer

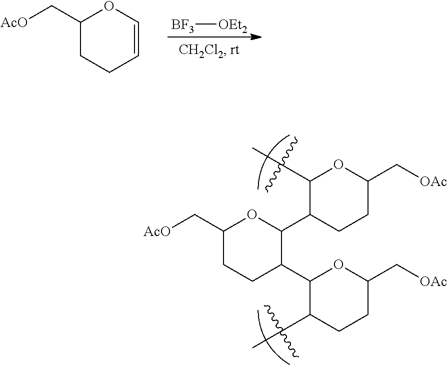

A small vial was loaded with acetic acid 3,4-dihydro-2H-pyran-2-ylmethyl ester (0.055 g, 0.35 mmol) and 2 mL of CH₂Cl₂ To this clear solution was added a diluted solution of BF₃—OEt₂ (0.005 mL, 0.035 mmol) in CH₂Cl₂. Within seconds, the clear solution began changing to yellow and then amber. After 24 hours, a brown precipitate had developed, and the supernatant was removed. The resultant oil was washed with triethylamine, hexanes and finally with cold water. The tan-white solid residue was dried in vacuo for 24 hours. Quantative yield. ¹H NMR (CD₃OD): broadened peaks 4.4-3.8 ppm, broadened peaks 2.0-1.0 ppm.

### Example 6

### Copolymer Synthesis of poly(acetic acid 3,4-dihydro-2H-pyran-2-ylmethyl ester) and 2-(4-isobutyl-phenyl)-propionic acid 3,4-dihydro-2H-pyran-2-ylmethyl ester (Drug Conjugate)

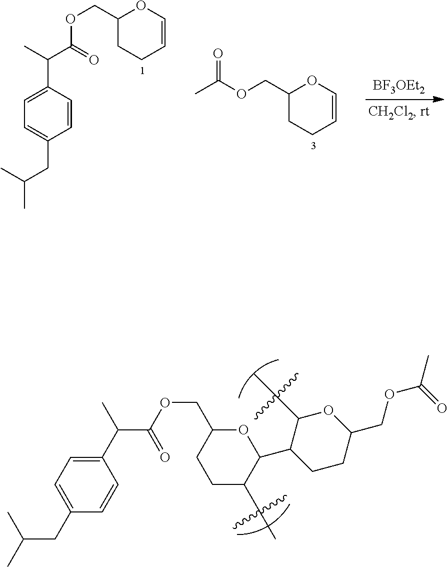 [SMILES: CC(C)Cc1ccc(C(C)C(=O)OCC2CCC=CO2)cc1 ; CC(=O)OCC1CCC=CO1 ; *C1CCC(COC(=O)C(C)c2ccc(CC(C)C)cc2)OC1C1CCC(COC(C)=O)OC1* ; "BF3OEt2"]

To a small vial was added acetic acid 3,4-dihydro-2H-pyran-2-ylmethyl ester (0.015 g, 0.096 mmol), 2-(4-isobutyl-phenyl)-propionic acid 3,4,-dihydro-2H-pyran-2-ylmethyl ester (0.033 mmol, 0.010g) and 2 mL of CH₂Cl₂. To this clear solution was added a diluted solution of BF₃—OEt₂ (0.002 mL, 0.013mmol) in CH₂Cl₂. Within seconds, the clear solution began changing color to yellow, then to amber. After 24 hours, the solvent was removed under reduced pressure. The resultant oil was washed with triethylamine, hexanes and finally with cold water. The tan-white solid residue was dried in vacuo for 24 hours. Quantative yield. ¹H NMR (CD₃OD): 7.2 ppm; 7.1 ppm; broadened peaks 4.4-3.8 ppm; 2.4 ppm; 2.1 ppm; overlapping broadened peaks 2.0-0.9 ppm.

### Example 7

### Synthesis of 2-methoxymethyl-3,4-dihydro-2H-pyran-3,4-diol Polymer with Coordinated ZnL₂ Complex (L=Cl)

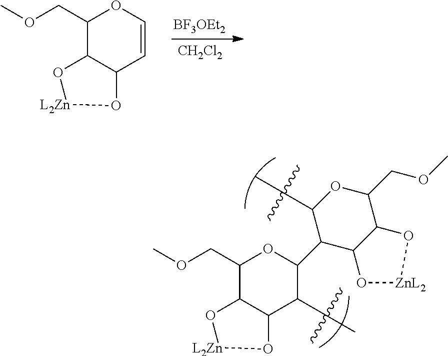

A small vial is charged with the monomer, acetic acid 2-methoxymethyl-3,4-dihydro-2H-pyran-3,4-diol (ZnL₂ complex, L=Cl), and 2 mL of CH₂Cl₂. To this solution is added a dilute solution of BF₃—OEt₂ (0.005 mL, 0.035 mmol) in CH₂Cl₂. After 24 hours, the solution is removed in vacuo to result in a white solid residue. The residue is washed with diethyl ether. The powder is characterized by ¹H NMR and IR spectroscopy.

### Example 8

### Synthesis of 1H-Pyrrole-2-carboxylic acid 3,4-dihydro-2H-2-ylmethyl ester Polymer with Coordinated AgL Complex (L=Pyridine)

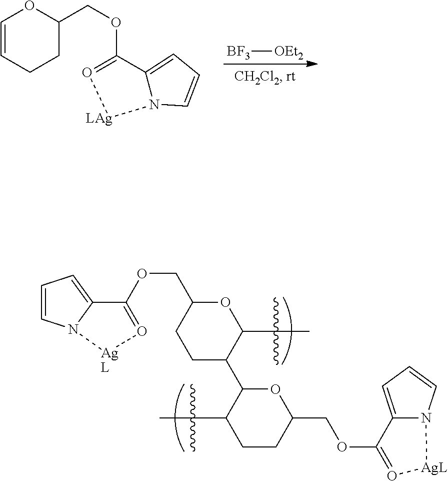

A small vial is loaded the monomer 1H-pyrrole-2-carboxylic acid 3,4-dihydro-2H-2-ylmethyl ester (AgL complex, L=pyridine) (0.050 grams, 0.127 mmols). The Ag-monomer complex is dissolved in CH₂Cl₂, followed by the addition of BF₃OEt₂ (in CH₂Cl₂). The reaction solution is stirred approximately 24 hours and purified by precipitation of the complex with diethyl ether. The white solid complex is washed with diethyl ether and dried in vacuo. The white powder is characterized by ¹H NMR and IR spectroscopy.

### Example 9

### Metal Coordination of SnCl₄ to Polymer Derived from the 3,4-dihydropyran-2H-pyran-2-methanol Monomer

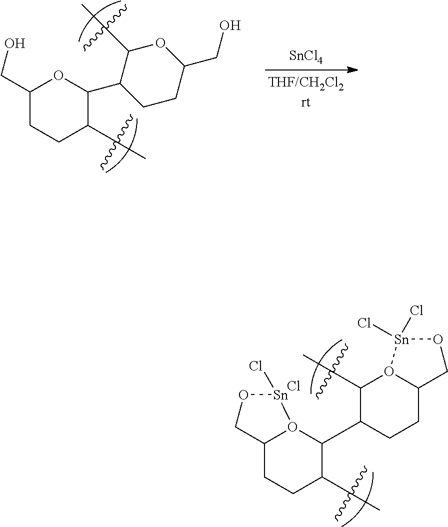

To a THF/CH₂Cl₂ solution of polymer comprised of the monomer 3,4-dihydropyran-2H-pyran-2-methanol (0.05 g) is added 1.2 equivalents of SnCl₄ (0.114 g, 0.53 mmol) dissolved in CH₂Cl₂ at rt. The solution is stirred for an additional 2 hours at rt. The Sn(IV)-adduct is precipitated out of solution with diethyl ether as a white solid. The white solid is washed with diethyl ether and dried in vacuo. The complex is characterized by NMR and IR spectroscopy.

### Example 10

### Synthesis of 1H-Pyrrole-2-carboxylic acid 3,4-dihydro-2H-2-ylmethyl ester Polymer with Coordinated Ag-Metronidazole Complex (Drug Conjugate)

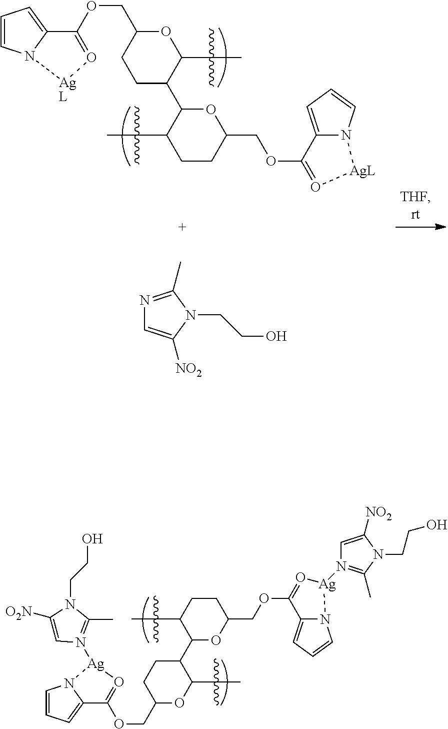

The Ag(L) coordinated polymer (0.050 g) prepared in Example 8 is dissolved in 2 mL of THF at rt. To this solution was added 1 equivalent of metronidazole. The mixture is allowed to stir for 12 hours at root temperature. The coordinated Ag-metronidazole containing polymer is precipated by the addition of diethyl ether to yield a white power. The powder is washed with diethylether and dried in vacuo. The complex is characterized by NMR and IR spectroscopy.

### Aspects of the Disclosure

Provided herein are oligomers and polymers comprising a plurality of optionally substituted carbon-linked tetrahydropyran rings. The oligomers and polymers are useful for treating pathological conditions in the oral cavity of an animal.

In one aspect, provided herein is an oligomer or polymer comprising a plurality of optionally substituted carbon-linked tetrahydropyran rings.

The oligomer of paragraph [00109] which comprises 2, 3, 4, 5, 6, 7, 8, 9, or 10 optionally substituted carbon-linked tetrahydropyran rings.

The polymer of paragraph [00109] which comprises at least 11 optionally substituted carbon-linked tetrahydropyran rings.

The polymer of paragraph [00109] which comprises at least 15 optionally substituted carbon-linked tetrahydropyran rings.

The polymer of paragraph [00109] which comprises at least 20 optionally substituted carbon-linked tetrahydropyran rings.

The polymer of paragraph [00109] which comprises at least 30 optionally substituted carbon-linked tetrahydropyran rings.

The polymer of any one of paragraphs [00111] to [00114] which comprises less than 500 optionally substituted carbon-linked tetrahydropyran rings.

The polymer of any one of paragraphs [00111] to [00114] which comprises less than 50 optionally substituted carbon-linked tetrahydropyran rings.

The oligomer or polymer of any one of paragraphs [00109] to [00116] wherein each optionally substituted carbon-linked tetrahydropyran ring has the structure (I):

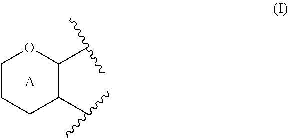

wherein ring A is optionally substituted, or a salt thereof.

The oligomer or polymer of any one of paragraphs [00109] to [00116] which comprises the structure (II):

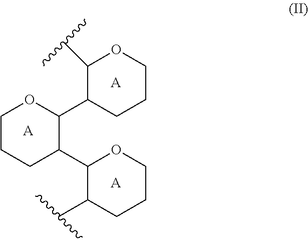

wherein each ring A is optionally substituted, or a salt thereof.

The oligomer or polymer of paragraph [00117] or [00118] wherein each ring A is optionally substituted with one or more groups independently selected from halo, —NO₂, —N(R^{b})₂, —CN, —C(O)—N(R^{b})₂, —O—R^{b}, —O—C(O)—R^{b}, —C(O)—R^{b}, —C(O)—O—R^{b}, —OS(O)₂(OR^{b}), —OP(O)(OR^{b})₂, —ONO₂, —N(R^{b})—C(O)—R^{b}, and —N(R^{b})—C(O)—N(R^{b})₂, C₁₋₈alkyl, C_{2 8}alkenyl, and C₂₋₈alkynyl, wherein any C₁₋₈alkyl, C_{2 8}alkenyl, and C₂₋₈alkynyl, is optionally substituted with one or more groups independently selected from the group consisting of halo, —NO₂, —N(R^{b})₂, —CN, —C(O)—N(R^{b})₂, —O—R^{b}, —O—C(O)—R^{b}, —C(O)—R^{b}, —C(O)—O—R^{b}, —OS(O)₂(OR^{b}), —OP(O)(OR^{b})₂, —ONO₂, —N(R^{b})—C(O)—R^{b}, and —N(R^{b})—C(O)—N(R^{b})₂; and each R^{b} is independently selected from the group consisting of hydrogen, C₁₋₆alkyl, C₂₋₆alkenyl, and C₂₋₆alkynyl, or a salt thereof.

The oligomer or polymer of paragraph [00117] or [00118] wherein each ring A has a structure independently selected from the group consisting of:

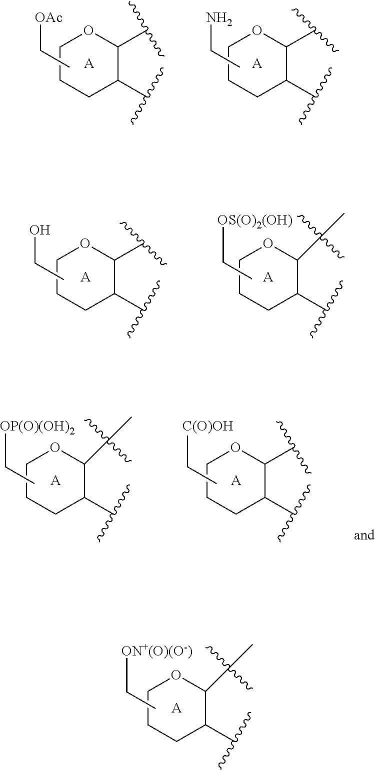

or a salt thereof.

The oligomer or polymer of paragraph [00117] or [00118] wherein each ring A has a structure independently selected from the group consisting of:

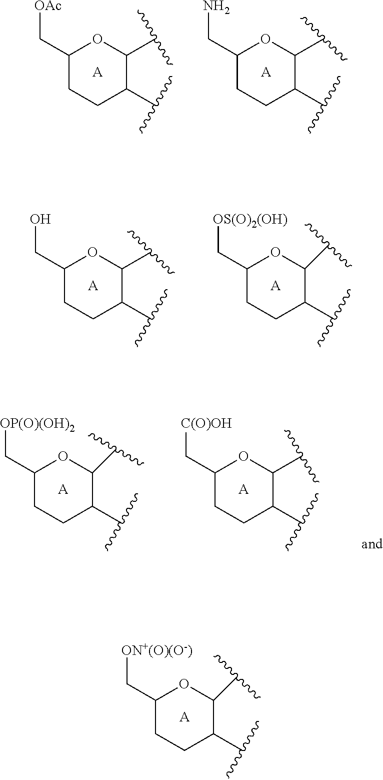

or a salt thereof.

The oligomer or polymer of any one of paragraphs [00109] to [00121] which further comprises a therapeutic agent.

The oligomer or polymer of paragraph [00122] wherein the therapeutic agent is dispersed in the polymer matrix.

The oligomer or polymer of paragraph [00122] wherein the therapeutic agent is covalently linked to the oligomer or polymer.

The oligomer or polymer of any one of paragraphs [00122] to [00124] wherein the therapeutic agent is selected from the group consisting of antimicrobials, anticancer agents and anesthetic agents

The oligomer or polymer of any one of paragraphs [00122] to [00124] wherein the therapeutic agent is selected from the group consisting of chlorhexidine, metronidazole, cisplatin, doxorubicin, lidocaine and benzocaine.

A mucoadhesive film comprising a polymer or oligomer as described in any one of paragraphs [00109] to [00126].

A method to treat a pathological condition in the oral cavity of an animal comprising administering an oligomer or polymer of any one of paragraphs [00109] to [00127].

The method of paragraph [00128] wherein the pathological condition is selected from oral mucositis, gum (periodontal) disease, a fungal infection, and oral cancer.

A guided tissue regeneration (GTR) film comprising an oligomer or polymer of any one of paragraphs [00109] to [0071].

The guided tissue regeneration (GTR) film of paragraph [00130] which has a thickness between about 100 and about 300 microns.

The guided tissue regeneration (GTR) film of paragraph [00130] or [00131] which is configured to regenerate tissue in the oral cavity.

A method for preparing an oligomer or polymer comprising: treating one or more monomers comprising a 3,4-dihydropyran ring with a catalyst to provide the oligomer or polymer. In some cases, the one or more monomers is protected with a well-known protecting group before the polymerization reaction, and deprotected after polymerization.

The method of paragraph [00133] wherein the catalyst comprises a transition metal.

The method of paragraph [00134] wherein the transition metal is selected from the group consisting of, palladium and platinum.

The method of paragraph [00135] wherein the transition metal is palladium.

The method of any one of paragraphs [00133] to [00136] wherein the catalyst comprises a coordinated solvent molecule.

The method of paragraph [00137] wherein the coordinated solvent molecule is selected from acetonitrile, THF and triflouroethanol.

The method of any one of paragraphs [00133] to [00138] wherein the treating is carried out in the presence of water and oxygen.

The method of any one of paragraphs [00133] to [00139] wherein each monomer is independently selected from compounds of the following formula:

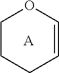

wherein ring A is optionally substituted.

The method of any one of paragraphs [00130] to [00139] wherein each monomer is independently selected from compounds of the following formula:

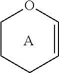

wherein ring A is optionally substituted with one or more groups independently selected from halo, —NO₂, —N(R^{b})₂, —CN, —C(O)—N(R^{b})₂, —O—R^{b}, —O—C(O)—R^{b}, —C(O)—R^{b}, —C(O)—O—R^{b}, —OS(O)₂(OR^{b}), —OP(O)(OR^{b})₂, —ONO₂, —N(R^{b})—C(O)—R^{b}, and —N(R^{b})—C(O)—N(R^{b})₂, C₁₋₈alkyl, C₂₋₈alkenyl, and C₂₋₈alkynyl, wherein any C₁₋₈alkyl, C₂₋₈alkenyl, and C₂₋₈alkynyl, is optionally substituted with one or more groups independently selected from the group consisting of halo, —NO₂, —N(R^{b})₂, —CN, —C(O)—N(R^{b})₂, —O—R^{b}, —O—C(O)—R^{b}, —C(O)—R^{b}, —C(O)—O—R^{b}, —OS(O)2(OR^{b}), —OP(O)(OR^{b})₂, —ONO₂, —N(R^{b})—C(O)—R^{b}, and —N(R^{b})—C(O)—N(R^{b})₂; and each R^{b} is independently selected from the group consisting of hydrogen, C₁₋₆alkyl, C₂₋₆alkenyl, and C_{2 6}alkynyl, or a salt thereof.

The method of any one of paragraphs [00130] to [00139] wherein each monomer is independently selected from the group consisting of:

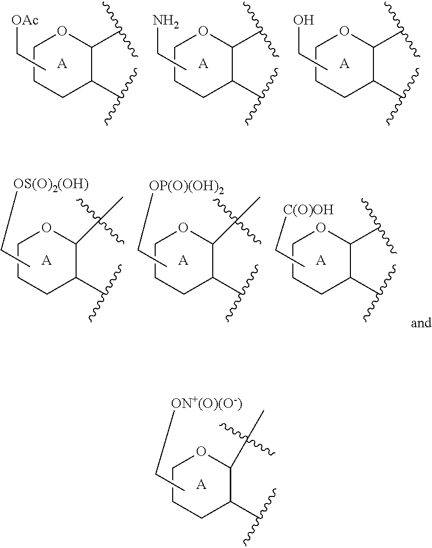

and salts thereof.

The method of any one of paragraphs [00130] to [00139] wherein each monomer is independently selected from the group consisting of:

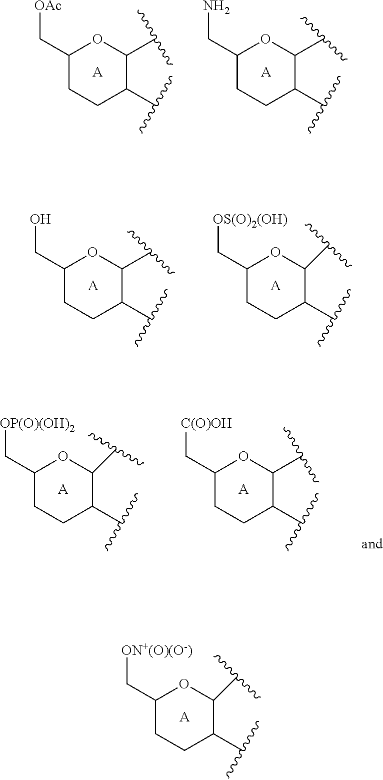

and salts thereof.

1. A polymer having a structure of Formula (II), or a pharmaceutically acceptable salt or solvate thereof: 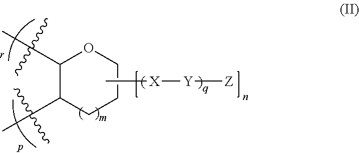
wherein:
p+r is 2 to 500;
m is 0 or 1;
when m is 0, then n is 1 or 2, and when m is 1 then n is 1, 2, or 3;
each q is 0 or 1;
each X independently is C₁₋₁₀alkylene, arylene, C₂₋₁₀polyalkyleneoxide, or absent;
each Y independently is O, S, or NR¹;
when q is 0, then each Z independently is C₁₋₁₀alkyl, C₀₋₁₀alkylene-C₃₋₈cycloalkyl, C₀₋₁₀alkylene-C₃₋₈heterocycloalkyl, C₀₋₁₀alkylene-aryl, C₀₋₁₀alkylene-heteroaryl, C₀₋₁₀alkylene-COOR¹, C₀₋₁₀alkylene-CN, C₀₋₁₀alkylene-NR¹₃⁺, C₀₋₁₀alkylene-ONO₂, C₀₋₁₀alkylene-OSO₂R², C₀₋₁₀alkylene-OPO₃H₂, C₂₋₁₀polyalkyleneoxide, C₂₋₁₀polyalkyleneoxide-C₃₋₈cycloalkyl, C₂₋₁₀polyalkyleneoxide-C₃₋₈heterocycloalkyl, C₂₋₁₀polyalkyleneoxide-aryl, C₂₋₁₀polyalkyleneoxide-heteroaryl, C₂₋₁₀polyalkyleneoxide-COOR¹, C₂₋₁₀polyalkyleneoxide-CN, C₂₋₁₀polyalkyleneoxide-NR¹₂, C₂₋₁₀polyalkyleneoxide-NR¹₃⁺, C₂₋₁₀polyalkyleneoxide-ONO₂, C₂₋₁₀polyalkyleneoxide-OSO₂R², C₂₋₁₀polyalkyleneoxide-OPO₃H₂, or 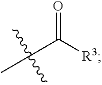
when q is 1, then each Z independently is H, C₀₋₁₀alkylene-C₃₋₈cycloalkyl, C₀₋₁₀alkylene-C₃₋₈heterocycloalkyl, C₀₋₁₀alkylene-aryl, C₀₋₁₀alkylene-heteroaryl, C₀₋₁₀alkylene-COOR¹, C₁₋₁₀alkylene-CN, C₁₋₁₀alkylene-OR¹, C₁₋₁₀alkylene-SR¹, C₁₋₁₀alkylene-NR¹₂, C₁₋₁₀alkylene-NR¹₃⁺, C₁₋₁₀alkylene-ONO₂, C₁₋₁₀alkylene-OSO₂R², C₁₋₁₀alkylene-OPO₃H₂, C₂₋₁₀polyalkyleneoxide, C₂₋₁₀polyalkyleneoxide-C₃₋₈cycloalkyl, C₂₋₁₀polyalkyleneoxide-C₃₋₈heterocycloalkyl, C₂₋₁₀polyalkyleneoxide-aryl, C₂₋₁₀polyalkyleneoxide-heteroaryl, C₂₋₁₀polyalkyleneoxide-COOR¹, C₂₋₁₀polyalkyleneoxide-CN, C₂₋₁₀polyalkyleneoxide-OR¹, C₂₋₁₀polyalkyleneoxide-SR¹, C₂₋₁₀polyalkyleneoxide-NR¹₂, C₂₋₁₀polyalkyleneoxide-NR¹₃⁺, C₂₋₁₀polyalkyleneoxide-ONO₂, C₂₋₁₀polyalkyleneoxide-OSO₂R², C₂₋₁₀polyalkyleneoxide-OPO₃H₂, or 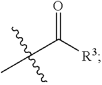
each R¹ independently is H, C₁₋₆alkyl, or aryl;
each R² independently is OR¹ or NR¹₂;
each R³ independently is C₁₋₁₀alkyl, C₁₋₁₀alkylene-C₃₋₈cycloalkyl, C₀₋₁₀alkylene-C₅₋₈heterocycloalkyl, C₀₋₁₀alkylene-aryl, C₀₋₁₀alkylene-heteroaryl, C₁₋₁₀alkylene-COOR⁴, C₀₋₁₀alkylene-OR⁴, C₀₋₁₀alkylene-SR⁴, C₀₋₁₀alkylene-NR¹₂, C₀₋₁₀alkylene-NR¹₃⁺, C₁₋₁₀alkylene-CN, C₁₋₁₀alkylene-NO₂, C₁₋₁₀alkylene-ONO₂, C₁₋₁₀alkylene-SO₂R², C₁₋₁₀alkylene-OSO₂R², C₀₋₁₀alkylene-PO₃H₂, C₀₋₁₀alkylene-OPO₃H₂, C₂₋₁₀polyalkyleneoxide, C₂₋₁₀polyalkyleneoxide-C₃₋₈cycloalkyl, C₂₋₁₀polyalkyleneoxide-C₅₋₈heterocycloalkyl, C₂₋₁₀polyalkyleneoxide-aryl, C₂₋₁₀polyalkyleneoxide-heteroaryl, C₂₋₁₀polyalkyleneoxide-COOR⁴, C₂₋₁₀polyalkyleneoxide-OR⁴, C₂₋₁₀polyalkyleneoxide-SR⁴, C₂₋₁₀polyalkyleneoxide-NR¹₂, C₂₋₁₀polyalkyleneoxide-NR¹₃⁺, C₂₋₁₀polyalkyleneoxide-CN, C₂₋₁₀polyalkyleneoxide—NO₂, C₂₋₁₀polyalkyleneoxide-SO₃R⁴, C₂₋₁₀polyalkyleneoxide-PO₃H₂, or CH(NR¹₂)R⁵;
each R⁴ independently is H, C₁₋₁₀alkyl, or C₂₋₁₀polyalkyleneoxide; and
each R⁵ independently is H, C₁₋₆alkyl, C₁₋₆alkylene-OR¹, C₁₋₆alkylene-SR¹, C₁₋₆alkylene-COOH, C₁₋₆alkylene-CONR¹₂, C₁₋₆alkylene-NR¹₂, C₁₋₆alkylene-NHC(NH₂)₂, C₁₋₆alkylene-aryl, or C₁₋₆alkylene-heteroaryl.

2. The polymer of claim 1, further comprising a metal cation complexed to at least one heteroatom of the polymer.

3-4. (canceled)

5. The polymer of claim 1, wherein m is 1.

6-8. (canceled)

9. The polymer of claim 1, wherein m is 0.

10-11. (canceled)

12. The polymer of claim 1, wherein q is 0.

13-21. (canceled)

22. The polymer of claim 1, wherein q is 1.

23-52. (canceled)

53. The polymer of claim 1, comprising a structure selected from the group consisting of: 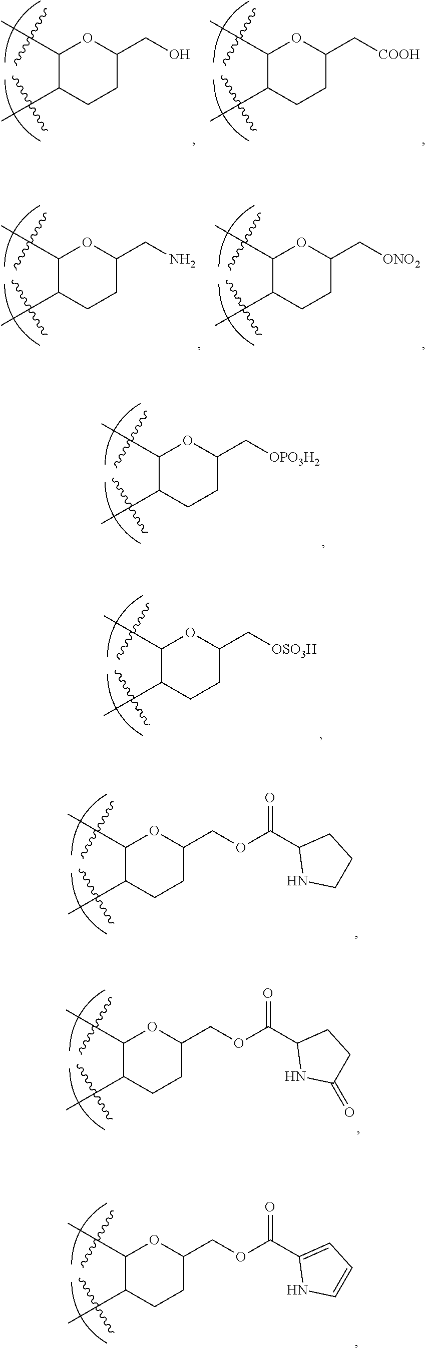 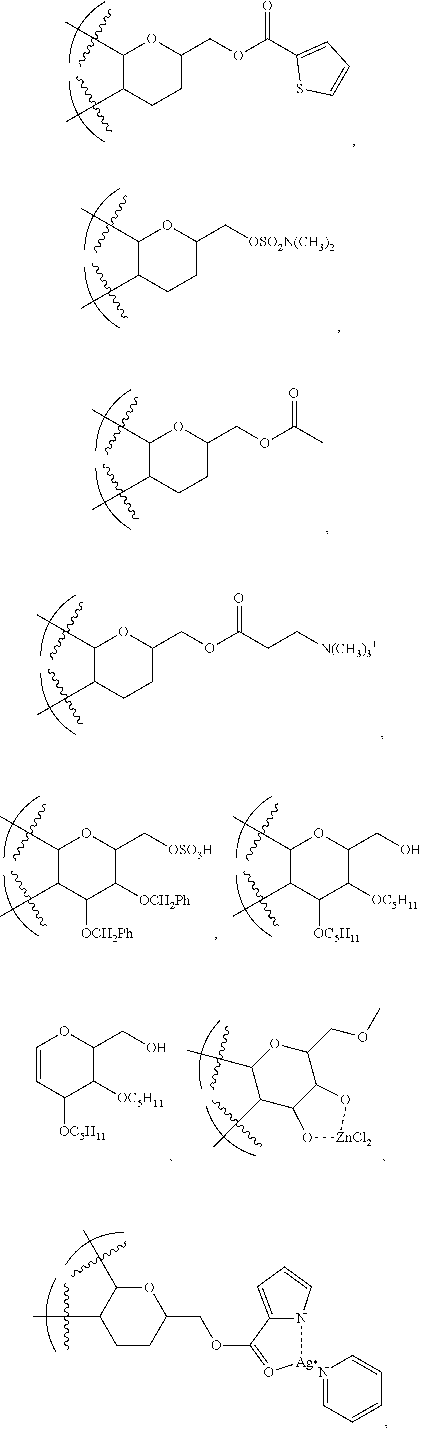 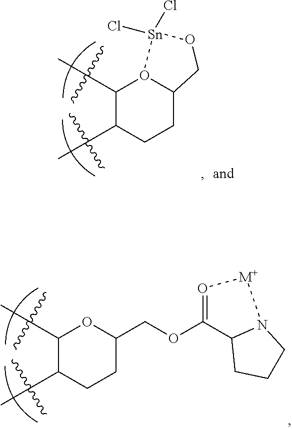
or a combination thereof, wherein M is Zn²⁺, Sn²⁺, Sn⁴⁺, or Ag⁺.

54. The polymer of claim 1, wherein p+r is at least 10.

55-56. (canceled)

57. The polymer of claim 1, further comprising one or more structural units of Formula (II′): 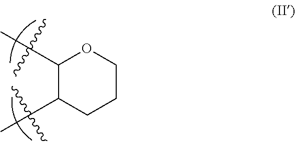
wherein the polymer comprises no more than 70% Formula (II′).

58. The polymer of claim 1, further comprising at least one monomer selected from the group consisting of ethylene, styrene, (C₀₋₂alkyl)acrylamide, (C₀₋₂alkyl)acrylate, (C₀₋₂alkyl)acrylic acid.

59. (canceled)

60. A monomer of Formula (I), or a pharmaceutically acceptable salt thereof: 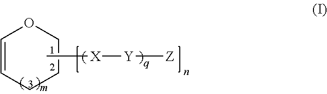
wherein:
m is 0 or 1;
when m is 0, then n is 1 or 2, and when m is 1 then n is 1, 2, or 3;
q is 0 or 1;
each X independently is C₁₋₁₀alkylene, arylene, or C₂₋₁₀polyalkyleneoxide;
each Y independently is O, S, or NR¹;
when q is 0, then each Z independently is C₀₋₁₀alkylene-C₃₋₈cycloalkyl, C₀₋₁₀alkylene-C₃₋₈heterocycloalkyl, C₀₋₁₀alkylene-aryl, C₀₋₁₀alkylene-heteroaryl, C₀₋₁₀alkylene-CN, C₀₋₁₀alkylene-NR¹₃⁺, C₀₋₁₀alkylene-ONO₂, C₀₋₁₀alkylene-OSO₂R², C₀₋₁₀alkylene-OPO₃H₂, C₂₋₁₀polyalkyleneoxide-C₃₋₈cycloalkyl, C₂₋₁₀polyalkyleneoxide-C₃₋₈heterocycloalkyl, C₂₋₁₀polyalkyleneoxide-aryl, C₂₋₁₀polyalkyleneoxide-heteroaryl, C₂₋₁₀polyalkyleneoxide-CN, C₂₋₁₀polyalkyleneoxide-NR¹₃⁺, C₂₋₁₀polyalkyleneoxide-ONO₂, C₂₋₁₀polyalkyleneoxide-OSO₂R², or C₂₋₁₀polyalkyleneoxide-OPO₃H₂;
when q is 1, then each Z independently is C₀₋₁₀alkylene-C₃₋₈cycloalkyl, C₀₋₁₀alkylene-C₃₋₈heterocycloalkyl, C₀₋₁₀alkylene-aryl, C₀₋₁₀alkylene-heteroaryl, C₀₋₁₀alkylene-COOR¹, C₁₋₁₀alkylene-CN, C₁₋₁₀alkylene-NR¹₂, C₁₋₁₀alkylene-NR¹₃⁺, C₁₋₁₀alkylene-ONO₂, C₁₋₁₀alkylene-OSO₂R², C₁₋₁₀alkylene-OPO₃H₂, C₂₋₁₀polyalkyleneoxide-C₃₋₈cycloalkyl, C₂₋₁₀polyalkyleneoxide-C₃₋₈heterocycloalkyl, C₂₋₁₀polyalkyleneoxide-aryl, C₂₋₁₀polyalkyleneoxide-heteroaryl, C₂₋₁₀polyalkyleneoxide-COOR¹, C₂₋₁₀polyalkyleneoxide-CN, C₂₋₁₀polyalkyleneoxide-OR¹, C₂₋₁₀polyalkyleneoxide-SR¹, C₂₋₁₀polyalkyleneoxide-NR¹₂, C₂₋₁₀polyalkyleneoxide-NR¹₃⁺, C₂₋₁₀polyalkyleneoxide-ONO₂, C₂₋₁₀polyalkyleneoxide-OSO₂R², C₂₋₁₀polyalkyleneoxide-OPO₃H₂, or 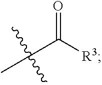
each R¹ independently is H, C₁₋₆alkyl, or aryl;
each R² independently is OR¹ or NR¹₂;
each R³ independently is C₁₋₁₀alkyl, C₀₋₁₀alkylene-C₃₋₈cycloalkyl, C₀₋₁₀alkylene-C₅₋₈heterocycloalkyl [Pro], C₀₋₁₀alkylene-aryl, C₀₋₁₀alkylene-heteroaryl [pyrrole], C₁₋₁₀alkylene-COOR⁴, C₀₋₁₀alkylene-OR⁴, C₀₋₁₀alkylene-SR⁴, C₀₋₁₀alkylene-NR¹₂, C₀₋₁₀alkylene-NR¹₃⁺, C₁₋₁₀alkylene-CN, C₁₋₁₀alkylene-NO₂, C₁₋₁₀alkylene-ONO₂, C₁₋₁₀alkylene-SO₂R², C₁₋₁₀alkylene-OSO₂R², C₁₋₁₀alkylene-PO₃H₂, C₁₋₁₀alkylene-OPO₃H₂, C₂₋₁₀polyalkyleneoxide, C₂₋₁₀polyalkyleneoxide-C₃₋₈cycloalkyl, C₂₋₁₀polyalkyleneoxide-C₅₋₈heterocycloalkyl, C₂₋₁₀polyalkyleneoxide-aryl, C₂₋₁₀polyalkyleneoxide-heteroaryl, C₂₋₁₀polyalkyleneoxide-COOR⁴, C₂₋₁₀polyalkyleneoxide-OR⁴, C₂₋₁₀polyalkyleneoxide-SR⁴, C₂₋₁₀polyalkyleneoxide-NR¹₂, C₂₋₁₀polyalkyleneoxide-NR¹₃⁺, C₂₋₁₀polyalkyleneoxide-CN, C₂₋₁₀polyalkyleneoxide—NO₂, C₂₋₁₀polyalkyleneoxide-SO₃R⁴, C₂₋₁₀polyalkyleneoxide-PO₃H₂, or CH(NR¹₂)R⁵;
each R⁴ independently is H, C₁₋₁₀alkyl, or C₂₋₁₀polyalkylene; and
ach R⁵ independently is H, C₁₋₆alkyl, C₁₋₆alkylene-OR¹, C₁₋₆alkylene-SR¹, C₁₋₆alkylene-COOH, C₁₋₆alkylene-CONR¹₂, C₁₋₆alkylene-NR¹₂, C₁₋₆alkylene-NHC(NH₂)₂, C₁₋₆alkylene-aryl, C₁₋₆alkylene-heteroaryl.

61. The monomer of claim 60 further comprising a metal cation complexed to at least one heteroatom of the monomer.

62-86. (canceled)

87. The monomer of claim 60 selected from the group consisting of: 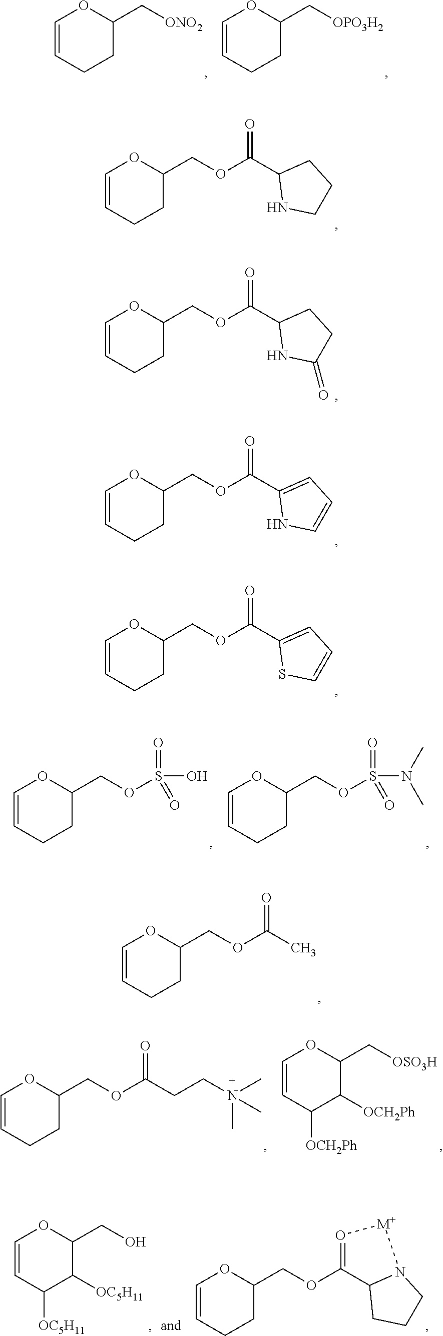
wherein M is Zn²⁺, Sn²⁺, Sn⁴⁺, or Ag⁺.

88. A drug conjugate, or a pharmaceutically acceptable salt thereof, comprising a therapeutic agent and the polymer of claim 1; wherein the therapeutic agent and monomer or polymer are attached through a linking group selected from the group consisting of an ester linkage, a thioester linkage, an amide linkage, a carbamate linkage, a carbonate linkage, and a metal ligand bond, and
the linking group is formed from the Z group of the monomer or polymer, and a reactive group on the therapeutic agent, such that:
(i) Z comprises a hydroxyl group, a thiol group, or an amino group, and the reactive group comprises a carboxylic acid or an activated carboxylic acid; or
(ii) Z comprises a carboxylic acid or an activated carboxylic acid, and the reactive group comprises a hydroxyl group, a thiol group, or an amino group; or
(iii) each of Z and the reactive group comprise a metal-binding ligand.

89-100. (canceled)

101. A method of treating a disease or condition in a subject, comprising contacting a non-keratinized surface in the oral cavity, oropharynx, or both of the subject with the polymer of claim 1 to provide a therapeutic beneficial effect on the disease or condition of the subject.

102-106. (canceled)

107. A drug conjugate, or a pharmaceutically acceptable salt thereof, comprising a therapeutic agent and the monomer of claim 60; wherein the therapeutic agent and monomer or polymer are attached through a linking group selected from the group consisting of an ester linkage, a thioester linkage, an amide linkage, a carbamate linkage, a carbonate linkage, and a metal ligand bond, and
the linking group is formed from the Z group of the monomer or polymer, and a reactive group on the therapeutic agent, such that:
(i) Z comprises a hydroxyl group, a thiol group, or an amino group, and the reactive group comprises a carboxylic acid or an activated carboxylic acid; or
(ii) Z comprises a carboxylic acid or an activated carboxylic acid, and the reactive group comprises a hydroxyl group, a thiol group, or an amino group; or
(iii) each of Z and the reactive group comprise a metal-binding ligand.
